(19) United States
(12) Patent Application Publication
(10) Pub. No.: US 2008/0199468 A1
(43) Pub. Date: Aug. 21, 2008

(54) Method For Diagnosing Colorectal Cancers

(75) Inventors: Yusuke Nakamura, Kanagawa (JP); Yoichi Furukawa, Kanagawa (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Oncotherapy Science, Inc., Kawasaki-shi (JP)

(21) Appl. No.: 10/589,594

(22) PCT Filed: Feb. 24, 2004
(86) PCT No.: PCT/JP04/02145

§ 371 Date: Jun. 11, 2007

(51) Int. Cl. A61K 39/395; C12Q 1/68; G01N 33/00; C40B 30/04; C07H 21/04; A61P 1/00; C12N 15/00; A61K 39/00; C12Q 1/34; A61K 31/70

(52) U.S. Cl. 424/138.1; 435/6; 436/86; 506/9; 435/18; 514/44; 424/277.1; 536/24.5; 435/320.1

(57) ABSTRACT

Objective methods for detecting and diagnosing colorectal cancer (CRC) are described herein. In one embodiment, the diagnostic method involves the determining an expression level of C10orf3 that discriminate between CRC and normal cell. The present invention further provides methods of screening for therapeutic agents useful in the treatment of CRC, methods of treating CRC and method of vaccinating a subject against CRC.

### FIELD OF THE INVENTION

The invention relates to methods of diagnosing colorectal cancers.

### BACKGROUND OF THE INVENTION

Colorectal cancer (CRC) is one of the most common solid tumors worldwide and nearly 940,000 individuals were diagnosed to have colon cancer in 2000 (1). Despite various advances in diagnosis and treatment of colorectal cancers, prognosis of patients with advanced stages results in high mortality. To improve their prognosis, development of a sensitive and specific diagnostic biomarker for detection of early-stage carcinomas and that of more effective but less harmful therapeutic drugs are desired. To achieve this goal, it is essential to understand better the molecular mechanisms of colorectal carcinogenesis.

Recent molecular studies have revealed that colorectal carcinogenesis involves accumulation of genetic alterations that include genetic changes in tumor suppressor genes and/or oncogenes including APC, p53, beta-catenin and Ki-ras (2-5). In addition, epigenetic events such as altered methylation (6) and loss of imprinting (7), and/or deregulated gene expression resulted from the genetic changes or other unknown mechanisms underlie the genesis of colorectal tumors. Since the effects on expression of various genes by each of these genetic and epigenetic changes are very complex, expression profiles of each cancer case are enormously different. Hence, for the comprehensive understanding of colorectal carcinogenesis, we need to investigate genome-wide expression profiles in colorectal tumors.

Analysis of expression profiles using cDNA microarrays is a promising method for identifying genes whose expression is altered by response to various physiological conditions or drugs, or pathological conditions (10, 11). This technology can also provide systematic expression profiles that may reflect the physiological or pathological phenotype of a subset of a cell population. This approach has proven to be useful for analyzing genes involved in various neoplasms and for detecting specific phenotypes (12-14). cDNA microarray technologies have enabled to obtain comprehensive profiles of gene expression in normal and malignant cells, and compare the gene expression in malignant and corresponding normal cells (Okabe et al., Cancer Res 61:2129-37 (2001); Kitahara et al., Cancer Res 61: 3544-9 (2001); Lin et al., Oncogene 21:4120-8 (2002); Hasegawa et al., Cancer Res 62:7012-7 (2002)). This approach enables to disclose the complex nature of cancer cells, and helps to understand the mechanism of carcinogenesis. Identification of genes that are deregulated in tumors can lead to more precise and accurate diagnosis of individual cancers, and to develop novel therapeutic targets (Bienz and Clevers, Cell 103:311-20 (2000)). To disclose mechanisms underlying tumors from a genome-wide point of view, and discover target molecules for diagnosis and development of novel therapeutic drugs, the present inventors have been analyzing the expression profiles of tumor cells using a cDNA microarray of 23040 genes (Okabe et al., Cancer Res 61:2129-37 (2001); Kitahara et al., Cancer Res 61:3544-9 (2001); Lin et al., Oncogene 21:4120-8 (2002); Hasegawa et al., Cancer Res 62:7012-7 (2002)). These efforts have pinpointed a number of genes, including ESTs, which appear to be up-regulated frequently in the cancer tissues compared with the corresponding non-cancerous cells.

Studies designed to reveal mechanisms of carcinogenesis have already facilitated identification of molecular targets for anti-tumor agents. For example, inhibitors of farnexyltransferase (FTIs) which were originally developed to inhibit the growth-signaling pathway related to Ras, whose activation depends on posttranslational farnesylation, has been effective in treating Ras-dependent tumors in animal models (He et al., Cell 99:335-45 (1999)). HER2/neu, a receptor that is over expressed in approximately 30% of breast cancers, mediates growth signals in response to specific ligand binding. Clinical trials on human using a combination or anti-cancer drugs and anti-HER2 monoclonal antibody, trastuzumab, have been conducted to antagonize the proto-oncogene receptor HER2/neu; and have been achieving improved clinical response and overall survival of breast-cancer patients (Molina et al., Cancer Res 61:4744-9 (2001); Lin et al., Cancer Res 61:6345-9 (2001)). A tyrosine kinase inhibitor, STI-571, which selectively inactivates bcr-abl fusion proteins, has been developed to treat chronic myelogenous leukemias wherein constitutive activation of bcr-abl tyrosine kinase plays a crucial role in the transformation of leukocytes (O'Dwyer et al., Curr Opin Oncol 12:594-7 (2000)). Agents of these kinds are designed to suppress oncogenic activity of specific gene products (Fujita et al., Cancer Res 61:7722-6 (2001)). Therefore, gene products commonly up-regulated in cancerous cells may serve as potential targets for developing novel anti-cancer agents.

It has been demonstrated that CD8+ cytotoxic T lymphocytes (CTLs) recognize epitope peptides derived from tumor-associated antigens (TAAs) presented on MHC Class I molecule, and lyse tumor cells. Since the discovery of MAGE family as the first example of TAAs, many other TAAs have been discovered using immunological approaches (Boon, Int J Cancer 54: 177-80 (1993); Boon and van der Bruggen, J Exp Med 183: 725-9 (1996); van der Bruggen et al., Science 254: 1643-7 (1991); Brichard et al., J Exp Med 178: 489-95 (1993); Kawakami et al., J Exp Med 180: 347-52 (1994)). Some of the discovered TAAs are now in the stage of clinical development as targets of immunotherapy. TAAs discovered so far include MAGE (van der Bruggen et al., Science 254: 1643-7 (1991)), gp100 (Kawakami et al., J Exp Med 180: 347-52 (1994)), SART (Shichijo et al., J Exp Med 187: 277-88 (1998)), and NY-ESO-1 (Chen et al., Proc Natl Acad Sci USA 94: 1914-8 (1997)). On the other hand, gene products which had been demonstrated to be specifically overexpressed in tumor cells, have been shown to be recognized as targets inducing cellular immune responses. Such gene products include p53 (Umano et al., Brit J Cancer 84: 1052-7 (2001)), HER2/neu (Tanaka et al., Brit J Cancer 84: 94-9 (2001)), CEA (Nukaya et al., Int J Cancer 80: 92-7 (1999)), and so on.

In spite of significant progress in basic and clinical research concerning TAAs (Rosenbeg et al., Nature Med 4: 321-7 (1998); Mukherji et al., Proc Natl Acad Sci USA 92: 8078-82 (1995); Hu et al., Cancer Res 56: 2479-83 (1996)), only limited number of candidate TAAs for the treatment of adenocarcinomas, including colorectal cancer, are available. TAAs abundantly expressed in cancer cells, and at the same time which expression is restricted to cancer cells would be promising candidates as immunotherapeutic targets. Further, identification of new TAAs inducing potent and specific antitumor immune responses is expected to encourage clinical use of peptide vaccination strategy in various types of cancer (Boon and can der Bruggen, J Exp Med 183: 725-9 (1996); van der Bruggen et al., Science 254: 1643-7 (1991); Brichard et al., J Exp Med 178: 489-95 (1993); Kawakami et al., J Exp Med 180: 347-52 (1994); Shichijo et al., J Exp Med 187: 277-88 (1998); Chen et al., Proc Natl Acad Sci USA 94: 1914-8 (1997); Harris, J Natl Cancer Inst 88: 1442-5 (1996); Butterfield et al., Cancer Res 59: 3134-42 (1999); Vissers et al., Cancer Res 59: 5554-9 (1999); van der Burg et al., J Immunol 156: 3308-14 (1996); Tanaka et al., Cancer Res 57: 4465-8 (1997); Fujie et al., Int J Cancer 80: 169-72 (1999); Kikuchi et al., Int J Cancer 81: 459-66 (1999); Oiso et al., Int J Cancer 81: 387-94 (1999)).

It has been repeatedly reported that peptide-stimulated peripheral blood mononuclear cells (PBMCs) from certain healthy donors produce significant levels of IFN-γ in response to the peptide, but rarely exert cytotoxicity against tumor cells in an HLA-A24 or -A0201 restricted manner in ⁵¹Cr-release assays (Kawano et al., Cancer Res 60: 3550-8 (2000); Nishizaka et al., Cancer Res 60: 4830-7 (2000); Tamura et al., Jpn J Cancer Res 92: 762-7 (2001)). However, both of HLA-A24 and HLA-A0201 are one of the popular HLA alleles in Japanese, as well as Caucasian (Date et al., Tissue Antigens 47: 93-101 (1996); Kondo et al., J Immunol 155: 4307-12 (1995); Kubo et al., J Immunol 152: 3913-24 (1994); Imanishi et al., Proceeding of the eleventh International Hictocompatibility Workshop and Conference Oxford University Press, Oxford, 1065 (1992); Williams et al., Tissue Antigen 49: 129 (1997)). Thus, antigenic peptides of carcinomas presented by these HLAs may be especially useful for the treatment of carcinomas among Japanese and Caucasian. Further, it is known that the induction of low-affinity CTL in vitro usually results from the use of peptide at a high concentration, generating a high level of specific peptide/MHC complexes on antigen presenting cells (APCs), which will effectively activate these CTL (Alexander-Miller et al., Proc Natl Acad Sci USA 93: 4102-7 (1996)).

### SUMMARY OF THE INVENTION

To search for potential molecular targets for development of novel anti-cancer drugs, we have been analyzing expression profiles of clinical samples from cancer patients using a genome-wide cDNA microarray. In experiments with colon-cancer cells, the gene encoding C10orf3 was among those that showed elevated expression. We showed that transfection of C10orf3 siRNAs suppressed growth of colon-cancer cells in culture.

The invention is based on the discovery of a pattern of gene expression of C10orf3 correlated with colorectal cancer (CRC). In the present invention, we reveal that C10orf3 is frequently up-regulated in colorectal cancer and that it is abundantly expressed in testis and slightly expressed in small intestine, colon, stomach, placenta, and ovary. Since its reduced expression in cancer cells resulted in their growth suppression, C10orf3 is likely to be essential for the growth of cancer cells. These data should help the better understanding of colorectal carcinogenesis and may provide clues for the development of novel therapeutic strategies of colon cancers.

Accordingly, the invention features a method of diagnosing or determining a predisposition to CRC in a subject by determining an expression level of C10orf3 in a patient derived biological sample, such as tissue sample. A normal cell is one obtained from colorectal tissue. An increase of the level of expression of the C10orf3 compared to a normal control level of the gene indicates that the subject suffers from or is at risk of developing CRC.

By normal control level is meant a level of gene expression detected in a normal, healthy individual or in a population of individuals known not to be suffering from CRC. A control level is a single expression pattern derived from a single reference population or from a plurality of expression patterns. For example, the control level can be a database of expression patterns from previously tested cells. A normal individual is one with no clinical symptoms of CRC.

An increase in the level of expression of C10orf3 detected in a test sample compared to a normal control level indicates the subject (from which the sample was obtained) suffers from or is at risk of developing CRC.

Gene expression is increased 10%, 25%, 50% compared to the control level. Alternately, gene expression is increased 0.1, 0.2, 1, 2, 5, 10 or more fold compared to the control level. Expression is determined by detecting hybridization, e.g., C10orf3 gene probe to a gene transcript of the patient-derived tissue sample.

The patient derived tissue sample is any tissue from a test subject, e.g., a patient known to or suspected of having CRC. For example, the tissue contains a colorectal cancer cell. For example, the tissue is a cell from colon.

The invention further provides methods of identifying an agent that inhibits the expression or activity of C10orf3 by contacting a test cell expressing C10orf3 with a test agent and determining the expression level or activity of the C10orf3. The test cell is a colon cell such as a colon cell from a colorectal cancer. A decrease of the level compared to a normal control level of the gene indicates that the test agent is an inhibitor of the C10orf3 and reduces a symptom of CRC.

The invention also provides a kit with a detection reagent which binds to C10orf3 nucleic acid sequences or which binds to a gene product encoded by the nucleic acid sequences.

Therapeutic methods include a method of treating or preventing CRC in a subject by administering to the subject an antisense composition. The antisense composition reduces the expression of a specific target gene, e.g., the antisense composition contains a nucleotide, which is complementary to a nucleic acid sequence of C10orf3. Another method includes the steps of administering to a subject an small interfering RNA (siRNA) composition. The siRNA composition reduces the expression of a nucleic acid of C10orf3. In yet another method, treatment or prevention of CRC in a subject is carried out by administering to a subject a ribozyme composition. The nucleic acid-specific ribozyme composition reduces the expression of a nucleic acid of C10orf3. Suitable mechanisms for in vivo expression of a gene of interest are known in the art.

The invention also includes vaccines and vaccination methods. For example, a method of treating or preventing CRC in a subject is carried out by administering to the subject a vaccine containing a polypeptide encoded by a nucleic acid of C10orf3 or an immunologically active fragment such a polypeptide. An immunologically active fragment is a polypeptide that is shorter in length than the full-length naturally-occurring protein and which induces an immune response. For example, an immunologically active fragment at least 8 residues in length and stimulates an immune cell such as a T cell or a B cell. Immune cell stimulation is measured by detecting cell proliferation, elaboration of cytokines (e.g., IL-2), or production of an antibody.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

One advantage of the methods described herein is that the disease is identified prior to detection of overt clinical symptoms. Other features and advantages of the invention will be apparent from the following detailed description, and from the claims.

### BRIEF DESCRIPTION OF THE FIGURES

FIG. 1(a) depicts relative expression ratios (cancer/non-cancer) of C10orf3 in primary 11 CRCs and nine adenomas examined by cDNA microarray. Its up-regulated expression (Cy3/Cy5 intensity ratio>2.0) was observed in all five CRCs and three out of four adenomas that passed through the cutoff filter (either Cy3 or Cy5 signals greater than 20,000). FIG. 1(b) is an illustration depicting genomic structure and the transcript of C10orf3 and its predicted protein motifs. An open box indicates an AAA (ATPase associated with a variety cellular activities) domain. FIG. 1(c) are photographs depicting semi-quantitative RT-PCR analysis of C10orf3 using additional 20 CRC cases. T, tumor tissue; N, normal tissue. Expression of GAPDH served as an internal control.

FIG. 2 is a photograph depicting multiple-tissue northern blot analysis of C10orf3.

FIG. 3(a) is a photograph depicting expression of C10orf3 protein in SW480, HCT116, HT29, SNUC4 and SNUC5 colon cancer and COS7 cells. The flag-tagged C10orf3 protein in COS7 cells transfected with pFlag-CMV-5a-C10orf3 served for a control. FIG. 3(b) is a photograph depicting expression of C10orf3 protein in four colon cancer and the corresponding normal tissues.

FIGS. 4(a) and (b) are photographs depicting subcellular localization of C10orf3. FIG. 4(a) shows COS7 cells transfected with pFlag-CMV-5a-C10orf3 were stained with anti-Flag monoclonal antibody and anti-C10orf3 polyclonal antibody. Flag-tagged protein was visualized by FITC conjugated secondary anti-mouse IgG antibody and Rhodamine conjugated anti-rabbit IgG antibody. DAPI was used for the counterstaining of nuclei. FIG. 4(b) shows immunocytochemical staining endogeneous C10orf3 protein in colon cancer cells. DAPI was used for the counterstaining of nuclei.

FIG. 5(a)-(c) depict effect of C10orf3-siRNAs on the expression of C10orf3 and viability of SW480 or HCT116 cells. FIG. 5(a) is a photograph depicting semi-quantitative RT-PCR analysis using RNA from cells transfected with plasmids expressing siRNAs to C10orf3-siRNA or control plasmid. FIGS. 5(b) and (c) depict effect of the C10orf3-siRNAs on the viability of SW480 or HCT116 cells measured by Giemsa's staining (b) and a MTT assay (c).

### DETAILED DESCRIPTION

The present invention is based in part on the discovery of elevated expression of C10orf3 in cells from colon of patients with CRC. The elevated gene expression was identified by using a comprehensive cDNA microarray system.

Using a cDNA microarray containing 23,040 genes, comprehensive gene-expression profiles of 20 patients were constructed previously. C10orf3 is expressed at high level in CRC patients. In the process candidate molecular marker was selected with the potential of detecting cancer-related proteins in serum or sputum of patients, and some potential targets for development of signal-suppressing strategies in human colorectal cancer were discovered. C10orf3 is shown as UPAAA1 on the cDNA microarray.

C10orf3 identified herein are used for diagnostic purposes as marker of CRC and as gene target, the expression of which is altered to treat or alleviate a symptom of CRC.

Unless indicated otherwise, “CRC” is meant to refer to any of the sequences disclosed herein.

By measuring expression of C10orf3 in a sample of cells, CRC is diagnosed. Similarly, by measuring the expression of C10orf3 in response to various agents, and agents for treating CRC can be identified.

The invention involves determining (e.g., measuring) the expression of C10orf3. Using sequence information provided by the GeneBank™ database entries for C10orf3 sequence, C10orf3 is detected and measured using techniques well known to one of ordinary skill in the art. For example, sequence within the sequence database entries corresponding to C10orf3, is used to construct probes for detecting C10orf3 RNA sequence in, e.g., northern blot hybridization analysis. As another example, the sequences can be used to construct primers for specifically amplifying C10orf3 in, e.g, amplification-based detection methods such as reverse-transcription based polymerase chain reaction.

Expression level of C10orf3 in the test cell population, e.g., a patient derived tissues sample is then compared to expression level of the C10orf3 in a reference population. The reference cell population includes one or more cells for which the compared parameter is known, i.e., CRC cells or non-CRC cells.

Whether or not a pattern of gene expression in the test cell population compared to the reference cell population indicates CRC or a predisposition thereto depends upon the composition of the reference cell population. For example, if the reference cell population is composed of non-CRC cells, a similar gene expression pattern in the test cell population and reference cell population indicates the test cell population is non-CRC. Conversely, if the reference cell population is made up of CRC cells, a similar gene expression profile between the test cell population and the reference cell population indicates that the test cell population includes CRC cells.

A level of expression of a CRC marker gene in a test cell population is considered altered in levels of expression if its expression level varies from the reference cell population by more than 1.0, 1.5, 2.0, 5.0, 10.0 or more fold from the expression level of the corresponding C10orf3 in the reference cell population.

Differential gene expression between a test cell population and a reference cell population is normalized to a control nucleic acid, e.g. a housekeeping gene. For example, a control nucleic acid is one which is known not to differ depending on the endometriotic or non-endometriotic state of the cell. Expression levels of the control nucleic acid in the test and reference nucleic acid can be used to normalize signal levels in the compared populations. Control genes include β-actin, glyceraldehyde 3-phosphate dehydrogenase or ribosomal protein P1.

The test cell population is compared to multiple reference cell populations. Each of the multiple reference populations may differ in the known parameter. Thus, a test cell population may be compared to a second reference cell population known to contain, e.g., CRC cells, as well as a second reference population known to contain, e.g., non-CRC cells (normal cells). The test cell is included in a tissue type or cell sample from a subject known to contain, or to be suspected of containing, CRC cells.

The test cell is obtained from a bodily tissue or a bodily fluid, e.g., biological fluid (such as blood or urine). For example, the test cell is purified from a tissue. Preferably, the test cell population comprises an epithelial cell. The epithelial cell is from tissue known to be or suspected to be a CRC.

Cells in the reference cell population are derived from a tissue type as similar to test cell. Optionally, the reference cell population is a cell line, e.g. a CRC cell line (positive control) or a normal non-CRC cell line (negative control). Alternatively, the control cell population is derived from a database of molecular information derived from cells for which the assayed parameter or condition is known.

The subject is preferably a mammal. The mammal can be, e.g., a human, non-human primate, mouse, rat, dog, cat, horse, or cow.

Expression of C10orf3 disclosed herein is determined at the protein or nucleic acid level using methods known in the art. For example, Northern hybridization analysis using probes which specifically recognize the sequence can be used to determine gene expression. Alternatively, expression is measured using reverse-transcription-based PCR assays, e.g., using primers specific for C10orf3. Expression is also determined at the protein level, i.e., by measuring the levels of polypeptide encoded by the gene product described herein, or biological activity thereof. Such methods are well known in the art and include, e.g., immunoassays based on antibodies to protein encoded by C10orf3. The biological activity of the protein encoded by the gene is also well known.

### Diagnosing CRC

CRC is diagnosed by measuring the level of expression of C10orf3 from a test population of cells, (i.e., a patient derived biological sample). Preferably, the test cell population contains an epithelial cell, e.g., a cell obtained from colon tissue. Gene expression is also measured from blood or other bodily fluids such as urine. Other biological samples can be used for measuring the protein level. For example, the protein level in the blood, or serum derived from subject to be diagnosed can be measured by immunoassay or biological assay.

Expression of C10orf3 is determined in the test cell or biological sample and compared to the expression of the normal control level. A normal control level is an expression profile of C10orf3 typically found in a population known not to be suffering from CRC. An increase of the level of expression in the patient derived tissue sample of C10orf3 indicates that the subject is suffering from or is at risk of developing CRC.

When C10orf3 is altered in the test population compared to the normal control level indicates that the subject suffers from or is at risk of developing CRC.

Identifying Agents that Inhibit C10orf3 Expression or Activity

An agent that inhibits the expression or activity of C10orf3 is identified by contacting a test cell population expressing C10orf3 with a test agent and determining the expression level or activity of C10orf3. A decrease of expression or activity in the presence of the agent compared to the normal control level (or compared to the level in the absence of the test agent) indicates the agent is an inhibitor of C10orf3 and useful to inhibit CRC.

The test cell population is any cell expressing C10orf3. For example, the test cell population contains an epithelial cell, such as a cell is or derived from colon. For example, the test cell is an immortalized cell line derived from colorectal cancer. Alternatively, the test cell is a cell, which has been transfected with C10orf3 or which has been transfected with a regulatory sequence (e.g. promoter sequence) from C10orf3 operably linked to a reporter gene.

### Assessing Efficacy of Treatment of CRC in a Subject

The differentially expressed C10orf3 identified herein also allow for the course of treatment of CRC to be monitored. In this method, a test cell population is provided from a subject undergoing treatment for CRC. If desired, test cell populations are obtained from the subject at various time points before, during, or after treatment. Expression of C10orf3, in the cell population is then determined and compared to a reference cell population which includes cells whose CRC state is known. The reference cells have not been exposed to the treatment.

If the reference cell population contains no CRC cells, a similarity in expression between C10orf3 in the test cell population and the reference cell population indicates that the treatment is efficacious. However, a difference in expression between C10orf3 in the test population and a normal control reference cell population indicates the less favorable clinical outcome or prognosis.

By “efficacious” is meant that the treatment leads to a reduction in expression of a pathologically up-regulated gene, increase in expression of a pathologically down-regulated gene or a decrease in size, prevalence, or metastatic potential of colorectal tumors in a subject. When treatment is applied prophylactically, “efficacious” means that the treatment retards or prevents CRC from forming or retards, prevents, or alleviates a symptom of clinical CRC. Assessment of colorectal tumors is made using standard clinical protocols.

Efficaciousness is determined in association with any known method for diagnosing or treating CRC. CRC is diagnosed for example, by identifying symptomatic anomalies. pos Selecting a Therapeutic Agent for Treating CRC that is Appropriate for a Particular Individual

Differences in the genetic makeup of individuals can result in differences in their relative abilities to metabolize various drugs. An agent that is metabolized in a subject to act as an anti-CRC agent can manifest itself by inducing a change in gene expression pattern in the subject's cells from that characteristic of an CRC state to a gene expression pattern characteristic of a non-CRC state. Accordingly, the differentially expressed C10orf3 disclosed herein allow for a putative therapeutic or prophylactic inhibitor of CRC to be tested in a test cell population from a selected subject in order to determine if the agent is a suitable inhibitor of CRC in the subject.

To identify an inhibitor of CRC, that is appropriate for a specific subject, a test cell population from the subject is exposed to a therapeutic agent, and the expression of C10orf3 is determined.

The test cell population contains a CRC cell expressing C10orf3. Preferably, the test cell is an epithelial cell. For example a test cell population is incubated in the presence of a candidate agent and the pattern of gene expression of the test sample is measured and compared to one or more reference profiles, e.g., a CRC reference expression profile or a non-CRC reference expression profile.

A decrease in expression of C10orf3 in a test cell population relative to a reference cell population containing CRC is indicative that the agent is therapeutic.

The test agent can be any compound or composition. For example, the test agents are immunomodulatory agents.

### Screening Assays for Identifying Therapeutic Agents

C10orf3 disclosed herein can also be used to identify candidate therapeutic agents for treating a CRC. The method is based on screening a candidate therapeutic agent to determine if it converts an expression profile of C10orf3 characteristic of a CRC state to a pattern indicative of a non-CRC state.

In the method, a cell is exposed to a test agent or a combination of test agents (sequentially or consequentially) and the expression of C10orf3 in the cell is measured. The expression level of C10orf3 in the test population is compared to expression level of C10orf3 in a reference cell population that is not exposed to the test agent.

An agent effective in suppressing expression of over-expressed genes is deemed to lead to a clinical benefit. Such compounds are further tested for the ability to prevent CRC growth.

In a further embodiment, the present invention provides methods for screening candidate agents which are potential targets in the treatment of CRC. As discussed in detail above, by controlling the expression levels or activities of marker gene, one can control the onset and progression of CRC. Thus, candidate agents, which are potential targets in the treatment of CRC, can be identified through screenings that use the expression levels and activities of marker gene as indices. In the context of the present invention, such screening may comprise, for example, the following steps:

a) contacting a test compound with a polypeptide encoded by a nucleic acid of C10orf3;

b) detecting the binding activity between the polypeptide and the test compound; and

c) selecting a compound that binds to the polypeptide

Alternatively, the screening method of the present invention may comprise the following steps:

a) contacting a candidate compound with a cell expressing C10orf3, and

selecting a compound that reduces the expression level of C10orf3.

Cells expressing marker gene include, for example, cell lines established from CRC; such cells can be used for the above screening of the present invention.

Alternatively, the screening method of the present invention may comprise the following steps:

a) contacting a test compound with a polypeptide encoded by a nucleic acid of C10orf3;

b) detecting the biological activity of the polypeptide of step (a); and

c) selecting a compound that suppresses the biological activity of the polypeptide encoded by a nucleic acid of C10orf3 in comparison with the biological activity detected in the absence of the test compound.

A protein required for the screening can be obtained as a recombinant protein using the nucleotide sequence of the marker gene. Based on the information of the marker gene, one skilled in the art can select any biological activity of the protein as an index for screening and a measurement method based on the selected biological activity. Preferably, cell proliferative activity of C10orf3 may be selected as the biological activity. The cell proliferative activity is detected by proliferation of cell line such as COS7 or NIH3T3.

Alternatively, the screening method of the present invention may comprise the following steps:

a) contacting a candidate compound with a cell into which a vector comprising the transcriptional regulatory region of C10orf3 and a reporter gene that is expressed under the control of the transcriptional regulatory region has been introduced

b) measuring the activity of said reporter gene; and

c) selecting a compound that reduces the expression level of said reporter gene, as compared to a control.

Suitable reporter genes and host cells are well known in the art. The reporter construct required for the screening can be prepared by using the transcriptional regulatory region of a marker gene. When the transcriptional regulatory region of a marker gene has been known to those skilled in the art, a reporter construct can be prepared by using the previous sequence information. When the transcriptional regulatory region of a marker gene remains unidentified, a nucleotide segment containing the transcriptional regulatory region can be isolated from a genome library based on the nucleotide sequence information of the marker gene.

The compound isolated by the screening is a candidate for drugs that inhibit the activity of the protein encoded by marker gene and can be applied to the treatment or prevention of CRC.

Moreover, compound in which a part of the structure of the compound inhibiting the activity of protein encoded by marker gene is converted by addition, deletion and/or replacement are also included in the compounds obtainable by the screening method of the present invention.

When administrating the compound isolated by the method of the invention as a pharmaceutical for humans and other mammals, such as mice, rats, guinea-pigs, rabbits, cats, dogs, sheep, pigs, cattle, monkeys, baboons, and chimpanzees, the isolated compound can be directly administered or can be formulated into a dosage form using known pharmaceutical preparation methods. For example, according to the need, the drugs can be taken orally, as sugar-coated tablets, capsules, elixirs and microcapsules, or non-orally, in the form of injections of sterile solutions or suspensions with water or any other pharmaceutically acceptable liquid. For example, the compounds can be mixed with pharmaceutically acceptable carriers or media, specifically, sterilized water, physiological saline, plant-oils, emulsifiers, suspending agents, surfactants, stabilizers, flavoring agents, excipients, vehicles, preservatives, binders, and such, in a unit dose form required for generally accepted drug implementation. The amount of active ingredients in these preparations makes a suitable dosage within the indicated range acquirable.

Examples of additives that can be mixed to tablets and capsules are, binders such as gelatin, corn starch, tragacanth gum and arabic gum; excipients such as crystalline cellulose; swelling agents such as corn starch, gelatin and alginic acid; lubricants such as magnesium stearate; sweeteners such as sucrose, lactose or saccharin; and flavoring agents such as peppermint, Gaultheria adenothrix oil and cherry. When the unit-dose form is a capsule, a liquid carrier, such as an oil, can also be further included in the above ingredients. Sterile composites for injections can be formulated following normal drug implementations using vehicles such as distilled water used for injections.

Physiological saline, glucose, and other isotonic liquids including adjuvants, such as D-sorbitol, D-mannose, D-mannitol, and sodium chloride, can be used as aqueous solutions for injections. These can be used in conjunction with suitable solubilizers, such as alcohol, specifically ethanol, polyalcohols such as propylene glycol and polyethylene glycol, non-ionic surfactants, such as Polysorbate 80™ and HCO-50.

Sesame oil or Soy-bean oil can be used as a oleaginous liquid and may be used in conjunction with benzyl benzoate or benzyl alcohol as a solubilizer and may be formulated with a buffer, such as phosphate buffer and sodium acetate buffer; a pain-killer, such as procaine hydrochloride; a stabilizer, such as benzyl alcohol and phenol; and an anti-oxidant. The prepared injection may be filled into a suitable ampule.

Methods well known to one skilled in the art may be used to administer the pharmaceutical composition of the present invention to patients, for example as intraarterial, intravenous, or percutaneous injections and also as intranasal, transbronchial, intramuscular or oral administrations. The dosage and method of administration vary according to the body-weight and age of a patient and the administration method; however, one skilled in the art can routinely select a suitable method of administration. If said compound is encodable by a DNA, the DNA can be inserted into a vector for gene therapy and the vector administered to a patient to perform the therapy. The dosage and method of administration vary according to the body-weight, age, and symptoms of the patient but one skilled in the art can suitably select them.

For example, although the dose of a compound that binds to the protein of the present invention and regulates its activity depends on the symptoms, the dose is about 0.1 mg to about 100 mg per day, preferably about 1.0 mg to about 50 mg per day and more preferably about 1.0 mg to about 20 mg per day, when administered orally to a normal adult (weight 60 kg).

When administering parenterally, in the form of an injection to a normal adult (weight 60 kg), although there are some differences according to the patient, target organ, symptoms and method of administration, it is convenient to intravenously inject a dose of about 0.01 mg to about 30 mg per day, preferably about 0.1 to about 20 mg per day and more preferably about 0.1 to about 10 mg per day. Also, in the case of other animals too, it is possible to administer an amount converted to 60 kgs of body-weight.

Assessing the Prognosis of a Subject with CRC

Also provided is a method of assessing the prognosis of a subject with CRC by comparing the expression of C10orf3 in a test cell population to the expression of the gene in a reference cell population derived from patients over a spectrum of disease stages. By comparing gene expression of C10orf3 in the test cell population and the reference cell population(s), or by comparing the pattern of gene expression over time in test cell populations derived from the subject, the prognosis of the subject can be assessed.

An increase of expression of C10orf3 compared to a normal control indicates less favorable prognosis. A decrease in expression of C10orf3 indicates a more favorable prognosis for the subject.

### Kits

The invention also includes a CRC-detection reagent, e.g., a nucleic acid that specifically binds to or identifies C10orf3 nucleic acids such as oligonucleotide sequences, which are complementary to a portion of a C10orf3 nucleic acid or antibodies which bind to proteins encoded by a C10orf3 nucleic acid. The reagents are packaged together in the form of a kit. The reagents are packaged in separate containers, e.g., a nucleic acid or antibody (either bound to a solid matrix or packaged separately with reagents for binding them to the matrix), a control reagent (positive and/or negative), and/or a detectable label. Instructions (e.g., written, tape, VCR, CD-ROM, etc.) for carrying out the assay are included in the kit. The assay format of the kit is a Northern hybridization or a sandwich ELISA known in the art.

For example, CRC detection reagent is immobilized on a solid matrix such as a porous strip to form at least one CRC detection site. The measurement or detection region of the porous strip may include a plurality of sites containing a nucleic acid. A test strip may also contain sites for negative and/or positive controls. Alternatively, control sites are located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of CRC present in the sample. The detection sites may be configured in any suitably detectable shape and are typically in the shape of a bar or dot spanning the width of a teststrip.

### Methods of Inhibiting CRC

The invention provides a method for treating or alleviating a symptom of CRC in a subject by decreasing expression or activity of C10orf3. Therapeutic compounds are administered prophylactically or therapeutically to subject suffering from (or susceptible to) developing CRC. Administration can be systemic or local. Such subjects are identified using standard clinical methods or by detecting an aberrant level of expression or activity of C10orf3. Therapeutic agents include inhibitors of cell proliferation.

The method includes decreasing the expression, or function, or both, of gene products of C10orf3. Expression is inhibited in any of several ways known in the art. For example, expression is inhibited by administering to the subject a nucleic acid that inhibits, or antagonizes, the expression of the over-expressed gene, e.g., an antisense oligonucleotide or small interfering RNA which disrupts expression of the over-expressed gene.

As noted above, antisense nucleic acids corresponding to the nucleotide sequence of C10orf3 can be used to reduce the expression level of the C10orf3. Antisense nucleic acids corresponding to the nucleotide sequence of C10orf3 that are up-regulated in CRC are useful for the treatment of CRC. Specifically, the antisense nucleic acids of the present invention may act by binding to the nucleotide sequence of C10orf3 or mRNA corresponding thereto, thereby inhibiting the transcription or translation of the gene, promoting the degradation of the mRNA, and/or inhibiting the expression of protein encoded by a nucleic acid of C10orf3, finally inhibiting the function of the proteins. The term “antisense nucleic acids” as used herein encompasses both nucleotides that are entirely complementary to the target sequence and those having a mismatch of nucleotide, so long as the antisense nucleic acids can specifically hybridize to the target sequences. For example, the antisense nucleic acids of the present invention include polynucleotides that have a homology of at least 70% or higher, preferably at 80% or higher, more preferably 90% or higher, even more preferably 95% or higher over a span of at least 15 continuous nucleotides. Algorithms known in the art can be used to determine the homology.

The antisense nucleic acid derivatives of the present invention act on cells producing the protein encoded by marker gene by binding to the DNA or mRNA encoding the protein, inhibiting their transcription or translation, promoting the degradation of the mRNA, and inhibiting the expression of the protein, thereby resulting in the inhibition of the protein function.

An antisense nucleic acid derivative of the present invention can be made into an external preparation, such as a liniment or a poultice, by mixing with a suitable base material which is inactive against the derivative.

Also, as needed, the derivatives can be formulated into tablets, powders, granules, capsules, liposome capsules, injections, solutions, nose-drops and freeze-drying agents by adding excipients, isotonic agents, solubilizers, stabilizers, preservatives, pain-killers, and such. These can be prepared by following known methods.

The antisense nucleic acids derivative is given to the patient by directly applying onto the ailing site or by injecting into a blood vessel so that it will reach the site of ailment. An antisense-mounting medium can also be used to increase durability and membrane-permeability. Examples are, liposomes, poly-L-lysine, lipids, cholesterol, lipofectin or derivatives of these.

The dosage of the antisense nucleic acid derivative of the present invention can be adjusted suitably according to the patient's condition and used in desired amounts. For example, a dose range of 0.1 to 100 mg/kg, preferably 0.1 to 50 mg/kg can be administered.

The antisense nucleic acids of the invention inhibit the expression of the protein of the invention and is thereby useful for suppressing the biological activity of a protein of the invention. Also, expression-inhibitors, comprising the antisense nucleic acids of the invention, are useful since they can inhibit the biological activity of a protein of the invention.

The antisense nucleic acids of present invention include modified oligonucleotides. For example, thioated nucleotides may be used to confer nuclease resistance to an oligonucleotide.

Also, a siRNA against marker gene can be used to reduce the expression level of the marker gene. By the term “siRNA” is meant a double stranded RNA molecule which prevents translation of a target mRNA. Standard techniques of introducing siRNA into the cell are used, including those in which DNA is a template from which RNA is transcribed. In the context of the present invention, the siRNA comprises a sense nucleic acid sequence and an anti-sense nucleic acid sequence against an upregulated marker gene, such as C10orf3. The siRNA is constructed such that a single transcript has both the sense and complementary antisense sequences from the target gene, e.g., a hairpin.

Binding of the siRNA to a transcript corresponding to C10orf3 in the target cell results in a reduction in the protein production by the cell. The length of the oligonucleotide is at least 10 nucleotides and may be as long as the naturally-occurring the transcript. Preferably, the oligonucleotide is 19-25 nucleotides in length. Most preferably, the oligonucleotide is less than 75, 50, 25 nucleotides in length. Examples of C10orf3 siRNA oligonucleotide which inhibit the expression in mammalian cells include the target sequence containing SEQ ID NO: 21. Furthermore, in order to enhance the inhibition activity of the siRNA, nucleotide “u” can be added to 3′end of the antisense strand of the target sequence. The number of “u”s to be added is at least 2, generally 2 to 10, preferably 2 to 5. The added “u”s form single strand at the 3′end of the antisense strand of the siRNA.

A C10orf3 siRNA is directly introduced into the cells in a form that is capable of binding to the mRNA transcripts. Alternatively, the DNA encoding the C10orf3 siRNA is in a vector.

Vectors are produced for example by cloning a C10orf3 target sequence into an expression vector operatively-linked regulatory sequences flanking the C10orf3 sequence in a manner that allows for expression (by transcription of the DNA molecule) of both strands (Lee, N. S., Dohjima, T., Bauer, G., Li, H., Li, M.-J., Ehsani, A., Salvaterra, P., and Rossi, J. (2002) Expression of small interfering RNAs targeted against HIV-1 rev transcripts in human cells. Nature Biotechnology 20: 500-505.). An RNA molecule that is antisense to C10orf3 mRNA is transcribed by a first promoter (e.g., a promoter sequence 3′ of the cloned DNA) and an RNA molecule that is the sense strand for the C10orf3 mRNA is transcribed by a second promoter (e.g., a promoter sequence 5′ of the cloned DNA). The sense and antisense strands hybridize in vivo to generate siRNA constructs for silencing of the C10orf3 gene. Alternatively, two constructs are utilized to create the sense and anti-sense strands of a siRNA construct. Cloned C10orf3 can encode a construct having secondary structure, e.g., hairpins, wherein a single transcript has both the sense and complementary antisense sequences from the target gene. A loop sequence consisting of an arbitrary nucleotide sequence can be located between the sense and antisense sequence in order to form the hairpin loop structure. Thus, the present invention also provides siRNA having the general formula 5′-[A]-[B]-[A′]-3′, wherein [A] is a ribonucleotide sequence corresponding to a sequence of nucleotides 1533-1551 (SEQ ID NO:21) of SEQ ID NO:1,

[B] is a ribonucleotide sequence consisting of 3 to 23 nucleotides, and

[A′] is a ribonucleotide sequence consisting of the complementary sequence of [A] The loop sequence may be preferably 3 to 23 nucleotide in length. The loop sequence, for example, can be selected from group consisting of following sequences (http://www.ambion.com/techlib/tb/tb_{—}506.html). In the siRNA of the present invention, nucleotide “u” can be added to the 3′end of [A′], in order to enhance the inhibiting activity of the siRNA. The number of “u”s to be added is at least 2, generally 2 to 10, preferably 2 to 5. Furthermore, loop sequence consisting of 23 nucleotides also provides active siRNA (Jacque, J.-M., Triques, K., and Stevenson, M. (2002) Modulation of HIV-1 replication by RNA interference. Nature 418: 435-438.).

AUG: Sui, G., Soohoo, C., Affar, E. B., Gay, F., Shi, Y., Forrester, W. C., and Shi, Y. (2002) A DNA vector-based RNAi technology to suppress gene expression in mammalian cells. Proc. Natl. Acad. Sci. USA 99(8): 5515-5520.

CCC, CCACC or CCACACC: Paul, C. P., Good, P. D., Winer, I., and Engelke, D. R. (2002) Effective expression of small interfering RNA in human cells. Nature Biotechnology 20: 505-508.

UUCG: Lee, N. S., Dohjima, T., Bauer, G., Li, H., Li, M.-J., Ehsani, A., Salvaterra, P., and Rossi, J. (2002) Expression of small interfering RNAs targeted against HIV-I rev transcripts in human cells. Nature Biotechnology 20: 500-505.

CTCGAG or AAGCUU: Editors of Nature Cell Biology (2003) Whither RNAi? Nat Cell Biol. 5:489-490.

UUCAAGAGA: Yu, J.-Y., DeRuiter, S. L., and Turner, D. L. (2002) RNA interference by expression of short-interfering RNAs and hairpin RNAs in mammalian cells. Proc. Natl. Acad. Sci. USA 99(9): 6047-6052.

For example, preferable siRNAs having hairpin structure of the present invention are shown below. In the following structure, the loop sequence can be selected from group consisting of AUG, CCC, UUCG, CCACC, CTCGAG, AAGCUU, CCACACC, and UUCAAGAGA. Preferable loop sequence is UUCAAGAGA (“ttcaagaga” in DNA). ggagagacugaaaacagag-[B]-cucuguuuucagucucucc (for target sequence of SEQ ID NO:21)

The regulatory sequences flanking the C10orf3 sequence are identical or are different, such that their expression can be modulated independently, or in a temporal or spatial manner. siRNAs are transcribed intracellularly by cloning the C10orf3 gene templates into a vector containing, e.g., a RNA pol III transcription unit from the small nuclear RNA (snRNA) U6 or the human H1 RNA promoter. For introducing the vector into the cell, transfection-enhancing agent can be used. FuGENE (Rochediagnostices), Lipofectamin 2000 (Invitrogen), Oligofectamin (Invitrogen), and Nucleofactor (Wako pure Chemical) are useful as the transfection-enhancing agent.

The nucleotide sequence of the siRNAs were designed using a siRNA design computer program available from the Ambion website (http://www.ambion.com/techlib/misc/siRNA_finder.html). The computer program selects nucleotide sequences for siRNA synthesis based on the following protocol.

Selection of siRNA Target Sites:

1. Beginning with the AUG start codon of the object transcript, scan downstream for AA dinucleotide sequences. Record the occurrence of each AA and the 3′ adjacent 19 nucleotides as potential siRNA target sites. Tuschl, et al. recommend against designing siRNA to the 5′ and 3′ untranslated regions (UTRs) and regions near the start codon (within 75 bases) as these may be richer in regulatory protein binding sites. UTR-binding proteins and/or translation initiation complexes may interfere with the binding of the siRNA endonuclease complex.

2. Compare the potential target sites to the human genome database and eliminate from consideration any target sequences with significant homology to other coding sequences. The homology search can be performed using BLAST, which can be found on the NCBI server at: www.ncbi.nlm.nih.gov/BLAST/

3. Select qualifying target sequences for synthesis. At Ambion, preferably several target sequences can be selected along the length of the gene for evaluation

Oligonucleotides and oligonucleotides complementary to various portions of C10orf3 mRNA were tested in vitro for their ability to decrease production of C10orf3 in tumor cells (e.g., using the COS7, NIH3T3 cell line and the HCT116 and SW948 colorectal cancer cell line) according to standard methods. A reduction in C10orf3 gene product in cells contacted with the candidate siRNA composition compared to cells cultured in the absence of the candidate composition is detected using C10orf3-specific antibodies or other detection strategies. Sequences which decrease production of C10orf3 in in vitro cell-based or cell-free assays are then tested for there inhibitory effects on cell growth. Sequences which inhibit cell growth in in vitro cell-based assay are test in in vivo in rats or mice to confirm decreased C10orf3 production and decreased tumor cell growth in animals with malignant neoplasms.

Also included in the invention are isolated nucleic acid molecules that include the nucleic acid sequence of target sequences, for example, nucleotides 1533-1551 (SEQ ID NO:21) of SEQ ID NO:1 or a nucleic acid molecule that is complementary to the nucleic acid sequence of nucleotides 1533-1551 (SEQ ID NO:21) of SEQ ID NO:1. As used herein, an “isolated nucleic acid” is a nucleic acid removed from its original environment (e.g., the natural environment if naturally occurring) and thus, synthetically altered from its natural state. In the present invention, isolated nucleic acid includes DNA, RNA, and derivatives thereof. When the isolated nucleic acid is RNA or derivatives thereof, base “t” should be replaced with “u” in the nucleotide sequences. As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof.

Complementary nucleic acid sequences hybridize under appropriate conditions to form stable duplexes containing few or no mismatches. Furthermore, the sense strand and antisense strand of the isolated nucleotide of the present invention, can form double stranded nucleotide or hairpin loop structure by the hybridization. In a preferred embodiment, such duplexes contain no more than 1 mismatch for every 10 matches. In an especially preferred embodiment, where the strands of the duplex are fully complementary, such duplexes contain no mismatches. The nucleic acid molecule is less than 2624 nucleotides in length. For example, the nucleic acid molecule is less than 500, 200, or 75 nucleotides in length. Also included in the invention is a vector containing one or more of the nucleic acids described herein, and a cell containing the vectors. The isolated nucleic acids of the present invention are useful for siRNA against C10orf3 or DNA encoding the siRNA. When the nucleic acids are used for siRNA or coding DNA thereof, the sense strand is preferably longer than 19 nucleotides, and more preferably longer than 21 nucleotides.

The antisense oligonucleotide or siRNA of the invention inhibit the expression of the polypeptide of the invention and is thereby useful for suppressing the biological activity of the polypeptide of the invention. Also, expression-inhibitors, comprising the antisense oligonucleotide or siRNA of the invention, are useful in the point that they can inhibit the biological activity of the polypeptide of the invention. Therefore, a composition comprising the antisense oligonucleotide or siRNA of the present invention are useful in treating a CRC.

Alternatively, function of gene product of the over-expressed gene is inhibited by administering a compound that binds to or otherwise inhibits the function of the gene products. For example, the compound is an antibody which binds to the over-expressed gene product.

The present invention refers to the use of antibodies, particularly antibodies against a protein encoded by an up-regulated marker gene, or a fragment of the antibody. As used herein, the term “antibody” refers to an immunoglobulin molecule having a specific structure, that interacts (i.e., binds) only with the antigen that was used for synthesizing the antibody (i.e., the up-regulated marker gene product) or with an antigen closely related to it. Furthermore, an antibody may be a fragment of an antibody or a modified antibody, so long as it binds to the protein encoded by the marker gene. For instance, the antibody fragment may be Fab, F(ab′)₂, Fv, or single chain Fv (scFv), in which Fv fragments from H and L chains are ligated by an appropriate linker (Huston J. S. et al. Proc. Natl. Acad. Sci. U.S.A. 85:5879-5883 (1988)). More specifically, an antibody fragment may be generated by treating an antibody with an enzyme, such as papain or pepsin. Alternatively, a gene encoding the antibody fragment may be constructed, inserted into an expression vector, and expressed in an appropriate host cell (see, for example, Co M. S. et al. J. Immunol. 152:2968-2976 (1994); Better M. and Horwitz A. H. Methods Enzymol. 178:476-496 (1989); Pluckthun A. and Skerra A. Methods Enzymol. 178:497-515 (1989); Lamoyi E. Methods Enzymol. 121:652-663 (1986); Rousseaux J. et al. Methods Enzymol. 121:663-669 (1986); Bird R. E. and Walker B. W. Trends Biotechnol. 9:132-137 (1991)).

An antibody may be modified by conjugation with a variety of molecules, such as polyethylene glycol (PEG). The present invention provides such modified antibodies. The modified antibody can be obtained by chemically modifying an antibody. These modification methods are conventional in the field.

Alternatively, an antibody may be obtained as a chimeric antibody, between a variable region derived from a nonhuman antibody and a constant region derived from a human antibody, or as a humanized antibody, comprising the complementarity determining region (CDR) derived from a nonhuman antibody, the frame work region (FR) derived from a human antibody, and the constant region. Such antibodies can be prepared by using known technologies.

Cancer therapies directed at specific molecular alterations that occur in cancer cells have been validated through clinical development and regulatory approval of anti-cancer drugs such as trastuzumab (Herceptin) for the treatment of advanced breast cancer, imatinib methylate (Gleevec) for chronic myeloid leukemia, gefitinib (Iressa) for non-small cell lung cancer (NSCLC), and rituximab (anti-CD20 mAb) for B-cell lymphoma and mantle cell lymphoma (Ciardiello F, Tortora G. A novel approach in the treatment of cancer: targeting the epidermal growth factor receptor. Clin Cancer Res. 2001 October;7(10):2958-70. Review.; Slamon D J, Leyland-Jones B, Shak S, Fuchs H, Paton V, Bajamonde A, Fleming T, Eiermann W, Wolter J, Pegram M, Baselga J, Norton L. Use of chemotherapy plus a monoclonal antibody against HER2 for metastatic breast cancer that overexpresses HER2. N Engl J Med. 2001 Mar. 15;344(11):783-92; Rehwald U, Schulz H, Reiser M, Sieber M, Staak J O, Morschhauser F, Driessen C, Rudiger T, Muller-Hermelink K, Diehl V, Engert A. Treatment of relapsed CD20+ Hodgkin lymphoma with the monoclonal antibody rituximab is effective and well tolerated: results of a phase 2 trial of the German Hodgkin Lymphoma Study Group. Blood. 2003 Jan. 15;101(2):420-424; Fang G, Kim C N, Perkins C L, Ramadevi N, Winton E, Wittmann S and Bhalla KN. (2000). Blood, 96, 2246-2253.). These drugs are clinically effective and better tolerated than traditional anti-cancer agents because they target only transformed cells. Hence, such drugs not only improve survival and quality of life for cancer patients, but also validate the concept of molecularly targeted cancer therapy. Furthermore, targeted drugs can enhance the efficacy of standard chemotherapy when used in combination with it (Gianni L. (2002). Oncology, 63 Suppl 1, 47-56; Klejman A, Rushen L, Morrione A, Slupianek A and Skorski T. (2002). Oncogene, 21, 5868-5876.). Therefore, future cancer treatments will probably involve combining conventional drugs with target-specific agents aimed at different characteristics of tumor cells such as angiogenesis and invasiveness.

These modulatory methods are performed ex vivo or in vitro (e.g., by culturing the cell with the agent) or, alternatively, in vivo (e.g., by administering the agent to a subject). The method involves administering a protein or combination of proteins or a nucleic acid molecule or combination of nucleic acid, molecules as therapy to counteract aberrant expression or activity of the differentially expressed genes.

Diseases and disorders that are characterized by increased (relative to a subject not suffering from the disease or disorder) levels or biological activity of the genes may be treated with therapeutics that antagonize (i.e., reduce or inhibit) activity of the over-expressed gene or genes. Therapeutics that antagonize activity are administered therapeutically or prophylactically.

Therapeutics that may be utilized include, e.g., (i) antibodies to the over-expressed sequence; (ii) antisense nucleic acids or nucleic acids that are “dysfunctional” (i.e., due to a heterologous insertion within the coding sequence of over-expressed sequence); (iii) small interfering RNA (siRNA); or (iv) modulators (i.e., inhibitors and antagonists that alter the interaction between an over -expressed polypeptide and its binding partner. The dysfunctional antisense molecules are utilized to “knockout” endogenous function of a polypeptide by homologous recombination (see, e.g., Capecchi, Science 244:1288-1292 1989).

Increased level can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of a gene whose expression is altered). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

Prophylactic administration occurs prior to the manifestation of overt clinical symptoms of disease, such that a disease or disorder is prevented or, alternatively, delayed in its progression.

Therapeutic methods include contacting a cell with an agent that modulates one or more of the activities of the gene product of the differentially expressed gene. An agent that modulates protein activity includes a nucleic acid or a protein, a naturally-occurring cognate ligand of these proteins, a peptide, a peptidomimetic, or other small molecule.

The present invention also relates to a method of treating or preventing CRC in a subject comprising administering to said subject a vaccine comprising a polypeptide encoded by a nucleic acid of C10orf3 or an immunologically active fragment of said polypeptide, or a polynucleotide encoding the polypeptide or the fragment thereof. An administration of the polypeptide induces an anti-tumor immunity in a subject. To inducing anti-tumor immunity, a polypeptide encoded by a nucleic acid of C10orf3 or an immunologically active fragment of said polypeptide, or a polynucleotide encoding the polypeptide is administered. The polypeptide or the immunologically active fragments thereof are useful as vaccines against CRC. In some cases the proteins or fragments thereof may be administered in a form bound to the T cell receptor (TCR) or presented by an antigen presenting cell (APC), such as macrophage, dendritic cell (DC), or B-cells. Due to the strong antigen presenting ability of DC, the use of DC is most preferable among the APCs.

In the present invention, vaccine against CRC refers to a substance that has the function to induce anti-tumor immunity upon inoculation into animals. According to the present invention, polypeptides encoded by a nucleic acid of C10orf3 or fragments thereof were suggested to be HLA-A24 or HLA-A*0201 restricted epitopes peptides that may induce potent and specific immune response against CRC cells expressing C10orf3. Thus, the present invention also encompasses method of inducing anti-tumor immunity using the polypeptides. In general, anti-tumor immunity includes immune responses such as follows:
induction of cytotoxic lymphocytes against tumors,
induction of antibodies that recognize tumors, and
induction of anti-tumor cytokine production.

Therefore, when a certain protein induces any one of these immune responses upon inoculation into an animal, the protein is decided to have anti-tumor immunity inducing effect. The induction of the anti-tumor immunity by a protein can be detected by observing in vivo or in vitro the response of the immune system in the host against the protein.

For example, a method for detecting the induction of cytotoxic T lymphocytes is well known. A foreign substance that enters the living body is presented to T cells and B cells by the action of antigen presenting cells (APCs). T cells that respond to the antigen presented by APC in antigen specific manner differentiate into cytotoxic T cells (or cytotoxic T lymphocytes; CTLs) due to stimulation by the antigen, and then proliferate (this is referred to as activation of T cells). Therefore, CTL induction by a certain peptide can be evaluated by presenting the peptide to T cell by APC, and detecting the induction of CTL. Furthermore, APC has the effect of activating CD4+ T cells, CD8+ T cells, macrophages, eosinophils, and NK cells. Since CD4+ T cells and CD8+ T cells are also important in anti-tumor immunity, the anti-tumor immunity inducing action of the peptide can be evaluated using the activation effect of these cells as indicators.

A method for evaluating the inducing action of CTL using dendritic cells (DCs) as APC is well known in the art. DC is a representative APC having the strongest CTL inducing action among APCs. In this method, the test polypeptide is initially contacted with DC, and then this DC is contacted with T cells. Detection of T cells having cytotoxic effects against the cells of interest after the contact with DC shows that the test polypeptide has an activity of inducing the cytotoxic T cells. Activity of CTL against tumors can be detected, for example, using the lysis of ⁵¹Cr-labeled tumor cells as the indicator. Alternatively, the method of evaluating the degree of tumor cell damage using ³H-thymidine uptake activity or LDH (lactose dehydrogenase)-release as the indicator is also well known.

Apart from DC, peripheral blood mononuclear cells (PBMCs) may also be used as the APC. The induction of CTL is reported that the it can be enhanced by culturing PBMC in the presence of GM-CSF and IL-4. Similarly, CTL has been shown to be induced by culturing PBMC in the presence of keyhole limpet hemocyanin (KLH) and IL-7.

The test polypeptides confirmed to possess CTL inducing activity by these methods are polypeptides having DC activation effect and subsequent CTL inducing activity. Therefore, polypeptides that induce CTL against tumor cells are useful as vaccines against tumors. Furthermore, APC that acquired the ability to induce CTL against tumors by contacting with the polypeptides are useful as vaccines against tumors. Furthermore, CTL that acquired cytotoxicity due to presentation of the polypeptide antigens by APC can be also used as vaccines against tumors. Such therapeutic methods for tumors using anti-tumor immunity due to APC and CTL are referred to as cellular immunotherapy.

Generally, when using a polypeptide for cellular immunotherapy, efficiency of the CTL-induction is known to increase by combining a plurality of polypeptides having different structures and contacting them with DC. Therefore, when stimulating DC with protein fragments, it is advantageous to use a mixture of multiple types of fragments.

Alternatively, the induction of anti-tumor immunity by a polypeptide can be confirmed by observing the induction of antibody production against tumors. For example, when antibodies against a polypeptide are induced in a laboratory animal immunized with the polypeptide, and when growth of tumor cells is suppressed by those antibodies, the polypeptide can be determined to have an ability to induce anti-tumor immunity.

Anti-tumor immunity is induced by administering the vaccine of this invention, and the induction of anti-tumor immunity enables treatment and prevention of CRC. Therapy against cancer or prevention of the onset of cancer includes any of the steps, such as inhibition of the growth of cancerous cells, involution of cancer, and suppression of occurrence of cancer. Decrease in mortality of individuals having cancer, decrease of tumor markers in the blood, alleviation of detectable symptoms accompanying cancer, and such are also included in the therapy or prevention of cancer. Such therapeutic and preventive effects are preferably statistically significant. For example, in observation, at a significance level of 5% or less, wherein the therapeutic or preventive effect of a vaccine against cell proliferative diseases is compared to a control without vaccine administration. For example, Student's t-test, the Mann-Whitney U-test, or ANOVA may be used for statistical analysis.

The above-mentioned protein having immunological activity or a vector encoding the protein may be combined with an adjuvant. An adjuvant refers to a compound that enhances the immune response against the protein when administered together (or successively) with the protein having immunological activity. Examples of adjuvants include cholera toxin, salmonella toxin, alum, and such, but are not limited thereto. Furthermore, the vaccine of this invention may be combined appropriately with a pharmaceutically acceptable carrier. Examples of such carriers are sterilized water, physiological saline, phosphate buffer, culture fluid, and such. Furthermore, the vaccine may contain as necessary, stabilizers, suspensions, preservatives, surfactants, and such. The vaccine is administered systemically or locally. Vaccine administration may be performed by single administration, or boosted by multiple administrations.

When using APC or CTL as the vaccine of this invention, tumors can be treated or prevented, for example, by the ex vivo method. More specifically, PBMCs of the subject receiving treatment or prevention are collected, the cells are contacted with the polypeptide ex vivo, and following the induction of APC or CTL, the cells may be administered to the subject. APC can be also induced by introducing a vector encoding the polypeptide into PBMCs ex vivo. APC or CTL induced in vitro can be cloned prior to administration. By cloning and growing cells having high activity of damaging target cells, cellular immunotherapy can be performed more effectively. Furthermore, APC and CTL isolated in this manner may be used for cellular immunotherapy not only against individuals from whom the cells are derived, but also against similar types of tumors from other individuals.

Furthermore, a pharmaceutical composition for treating or preventing a cell proliferative disease, such as cancer, comprising a pharmaceutically effective amount of the polypeptide of the present invention is provided. The pharmaceutical composition may be used for raising anti tumor immunity.

### Pharmaceutical Compositions for Inhibiting CRC

Pharmaceutical formulations include those suitable for oral, rectal, nasal, topical (including buccal and sub-lingual), vaginal or parenteral (including intramuscular, sub-cutaneous and intravenous) administration, or for administration by inhalation or insufflation. Preferably, administration is intravenous. The formulations are optionally packaged in discrete dosage units.

Pharmaceutical formulations suitable for oral administration include capsules, cachets or tablets, each containing a predetermined amount of the active ingredient. Formulations also include powders, granules or solutions, suspensions or emulsions. The active ingredient is optionally administered as a bolus electuary or paste. Tablets and capsules for oral administration may contain conventional excipients such as binding agents, fillers, lubricants, disintegrant or wetting agents. A tablet may be made by compression or molding, optionally with one or more formulational ingredients. Compressed tablets may be prepared by compressing in a suitable machine the active ingredients in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may be coated according to methods well known in the art. Oral fluid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives. The tablets may optionally be formulated so as to provide slow or controlled release of the active ingredient therein. A package of tablets may contain one tablet to be taken on each of the month.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Formulations for rectal administration include suppositories with standard carriers such as cocoa butter or polyethylene glycol. Formulations for topical administration in the mouth, for example buccally or sublingually, include lozenges, which contain the active ingredient in a flavored base such as sucrose and acacia or tragacanth, and pastilles comprising the active ingredient in a base such as gelatin and glycerin or sucrose and acacia. For intra-nasal administration the compounds of the invention may be used as a liquid spray or dispersible powder or in the form of drops. Drops may be formulated with an aqueous or non-aqueous base also comprising one or more dispersing agents, solubilizing agents or suspending agents.

For administration by inhalation the compounds are conveniently delivered from an insufflator, nebulizer, pressurized packs or other convenient means of delivering an aerosol spray. Pressurized packs may comprise a suitable propellant such as dichlorodifluoromethane, trichlorofluoromethane, dichiorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflators.

Other formulations include implantable devices and adhesive patches; which release a therapeutic agent.

When desired, the above described formulations, adapted to give sustained release of the active ingredient, may be employed. The pharmaceutical compositions may also contain other active ingredients such as antimicrobial agents, immunosuppressants or preservatives.

It should be understood that in addition to the ingredients particularly mentioned above, the formulations of this invention may include other agents conventional in the art having regard to the type of formulation in question, for example, those suitable for oral administration may include flavoring agents.

Preferred unit dosage formulations are those containing an effective dose, as recited below, or an appropriate fraction thereof, of the active ingredient.

For each of the aforementioned conditions, the compositions, e.g., polypeptides and organic compounds are administered orally or via injection at a dose of from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg.

The dose employed will depend upon a number of factors, including the age and sex of the subject, the precise disorder being treated, and its severity. Also the route of administration may vary depending upon the condition and its severity.

The invention will be further described in the following examples, which do not limit the scope of the invention described in the claims.

### EXAMPLE 1

### Materials and Methods

Genome-wide cDNA Microarray

In the present invention, we used our original genome-wide cDNA microarray with 23040 genes. Briefly, DNase I treated total RNA extracted from the microdissected tissue was amplified with Ampliscribe T7 Transcription Kit (Epicentre Technologies, Madison, Wis., USA) and labeled during reverse transcription with Cy-dye (Amersham Biosciences Corp., Piscataway, N.J., USA); RNA from non-cancerous tissue with Cy5 and RNA from tumor with Cy3. Labeled RNA was hybridized to the cDNA microarray slides using Automated Slide Processor (Amersham Biosciences Corp., Piscataway, N.J., USA). The Hybridized slides were scanned with Array Scanner (Amersham Biosciences Corp., Piscataway, N.J., USA) and fluorescence intensity of Cy5 and Cy3 for each target spot was generated by Array Vision software (Amersham Biosciences Corp., Piscataway, N.J., USA). After subtraction of background signal, the duplicate values were averaged for each spot. Then, all fluorescence intensities on a slide were normalized to adjust the mean Cy5 and Cy3 intensity of 52 housekeeping genes for each slide.

### Cell Lines

COS7 cells and human colon cancer cell lines, HCT116, and SW480 were obtained from the American Type Culture Collection (ATCC), while human colon cancer cell lines, HT29, SNUC4 and SNUC5, were obtained from the Korea cell-line bank. All cells were grown in monolayers in appropriate media: Dulbecco's modified Eagle's medium for COS7 and RPMI1640 for HT29, SNUC4 and SNUC5; McCoy's 5A medium for HCT116; Leibovitz's L-15 for SW480; All media were supplemented with 10% fetal bovine serum and 1% antibiotic/antimycotic solution (Sigma-Aldrich Corp., St. Louis, Mo., USA).

### RNA Preparation and RT-PCR

Total RNA was extracted with a Qiagen RNeasy kit (Qiagen Inc., Valencia, Calif., USA) or Trizol reagent (Life Technologies, Inc.) according to the manufacturers' protocols. Ten-microgram aliquots of total RNA were reversely transcribed for single-stranded cDNAs using poly dT₁₂₋₁₈ primer (Amersham Biosciences Corp., Piscataway, N.J., USA) with Superscript II reverse transcriptase (Life Technologies). Each single-stranded cDNA preparation was diluted for subsequent PCR amplification by standard RT-PCR experiments carried out in 20-μl volumes of PCR buffer (TAKARA). Amplification proceeded for 4 min at 94° C. for denaturing, followed by 20 (for GAPDH), or 35 for C10orf3) cycles of 94° C. for 30 s, 60° C. for 30 s, and 72° C. for 45 s, in the GeneAmp PCR system 9700 (Perkin-Elmer, Foster City, Calif.). Primer sequences were;

(SEQ ID No; 3)

| | |
|---|---|
| for GAPDH: | forward, 5′-ACAACAGCCTCAAGATCATCAG-3′ |
| and | |

(SEQ ID No; 4)

reverse, 5′-GGTCCACCACTGACACGTTG-3′;

(SEQ ID No; 5)

| | |
|---|---|
| for | forward, 5′-AGAGATCCGAAGAGCTCTTATCT-3′ |
| C10orf3: | |
| and | |

(SEQ ID No; 6)

reverse, 5′-GATGCTCAGTGGCTGGATACT-3′.

### Northern-Blot Analysis

Human multiple-tissue blots (Clontech, Palo Alto, Calif., USA) were hybridized with a ³²P-labeled PCR product of C10orf3. Pre-hybridization, hybridization and washing were performed according to the supplier's recommendations. The blots were autoradiographed with intensifying screens at −80° C. for 5 days.

Construction of Plasmids Expressing C10orf3

The entire coding region of C10orf3 was amplified by RT-PCR using a gene specific primer set;

5′-CGAAAGCTTCAGAGATGTCTTCCA-3′ (SEQ ID No; 7)
(forward)
and
5′-AATGGATCCCTTTGAACAGTATTCCAC-3′ (SEQ ID No; 8)
(reverse).

The PCR product was cloned into appropriate cloning sites of pcDNA3.1 (Invitrogen Corp., Carlsbad, Calif., USA), pFLAG-CMV-5a (Sigma-Aldrich Corp., St. Louis, Mo., USA), pcDNA3.1myc/His (Invitrogen Corp., Carlsbad, Calif., USA), Similarly RT-PCR product using a set of primers,

5′-ATAGAATTCATGTCTTCCAGAAGTAC-3′ (SEQ ID No; 9) (forward) and

5′-TATCTCGAGCTTTGAACAGTAT-3′ (SEQ ID No; 10) (reverse) was cloned into pET28a (Novagen, Madison, Wis., USA) vector.

Preparation of Polyclonal Antibody to C10orf3

Recombinant His-tagged C10orf3 protein was produced in E. coli and purified from the cells using TALON® Superflow Metal Affinity Resin according to the manufacturer's recommendations (BD Biosciences Clontech, Palo Alto, Calif., USA). The recombinant protein was inoculated for the immunization of rabbits. The polyclonal antibody to C10orf3 was purified from the sera Proteins were separated by 10% SDS-PAGE and immunoblotted with the antibody. HRP-conjugated goat anti-rabbit IgG (Santa Cruz Biotechnology, Santa Cruz, Calif.) served as the second antibody for the ECL Detection System (Amersham Pharmacia Biotech, Piscataway, N.J.). Immunoblotting with the anti-C10orf3 antibody showed a 54 kD band of flag-tagged C10orf3, which was identical pattern to that detected using anti-flag antibody.

### Immunohistochemical Staining

COS7 cells transfected with pFLAG-CMV-5a-C10orf3 and colon cancer cell lines, SW480, HCT116 and SNUC5 were fixed with PBS containing 4% paraformaldehyde for 15 min, then rendered permeable with PBS containing 0.1% Triton X-100 for 2.5 min at RT. Subsequently the cells were covered with 3% BSA in PBS for 10 min at RT to block non-specific hybridization. COS7 cells transfected with pFLAG-CMV-5a-C10orf3 were double stained with mouse anti-flag monoclonal antibody (Sigma-Aldrich Corp., St. Louis, Mo., USA) at 1:1000 dilution and rabbit anti-C10orf3 polyclonal antibody at 1:1000 dilution. Subsequently, the cells were visualized after incubation with FITC-conjugated anti-mouse second antibody (WAKO, Osaka) and Rhodamine-conjugated anti-rabbit second antibody (WAKO, Osaka). SW480, HCT116 and SNUC5 cells were stained with rabbit anti-C10orf3 antibody at 1:2000 dilution and visualized with Rhodamine-conjugated anti-rabbit second antibody (WAKO, Osaka). Nucleus were counter-stained with 4′,6′-diamidine-2′-phenylindole dihydrochloride (DAPI). Fluorescent images were obtained under a spectral confocal scanning system (Leica).

Construction of Plasmid Expressing C10orf3-siRNA and its Gene Silencing Effect

To prepare plasmid vector expressing short interfering RNA (siRNA), we amplified the genomic fragment of HI RNA gene containing its promoter region by PCR using a set of primers, 5′-TGGTAGCCAAGTGCAGGTTATA-3′ (SEQ ID No; 11), and 5′-CCAAAGGGTTTCTGCAGTTTCA-3′ (SEQ ID No; 12) and human placental DNA as a template. The product was purified and cloned into pCR2.0 plasmid vector using a TA cloning kit according to the supplier's protocol (Invitrogen). The BamHI and XhoI fragment containing H1RNA was into pcDNA3.1(+) between nucleotides 56 and 1257, and the fragment was amplified by PCR using

(SEQ ID No; 13)

5′-TGCGGATCCAGAGCAGATTGTACTGAGAGT-3′
and

(SEQ ID No; 14)

5′-CTCTATCTCGAGTGAGGCGGAAAGAACCA-3′,

The ligated DNA became the template for PCR amplification with primers,

(SEQ ID No; 15)

5′-TTTAAGCTTGAAGACCATTTTTGGAAAAAAAAAAAAAAAAAAAAA
A-3′
and

(SEQ ID No; 16)

5′-TTTAAGCTTGAAGACATGGGAAAGAGTGGTCTCA-3′.

The product was digested with HindIII, and subsequently self-ligated to produce psiH1 BX3.0 vector plasmid having a nucleotide sequence shown in SEQ ID NO: 23.

The DNA flagment encoding siRNA was inserted into the GAP at nucleotide 489-492 as indicated (−) in the following plasmid sequence (SEQ ID No: 23)

GACGGATCGGGAGATCTCCCGATCCCCTATGGTGCACTCTCAGTACAATC
TGCTCTGGATCCACTAGTAACGGCCGCCAGTGTGCTGGAATTCGGCTTGG
TAGCCAAGTGCAGGTTATAGGGAGCTGAAGGGAAGGGGGTCACAGTAGGT
GGCATCGTTCCTTTCTGACTGCCCGCCCCCCGCATGCCGTCCCGCGATAT
TGAGCTCCGAACCTCTCGCCCTGCCGCCGCCGGTGCTCCGTCGCCGCCGC
GCCGCCATGGAATTCGAACGCTGACGTCATCAACCCGCTCCAAGGAATCG
CGGGCCCAGTGTCACTAGGCGGGAACACCCAGCGCGCGTGCGCCCTGGCA
GGAAGATGGCTGTGAGGGACAGGGGAGTGGCGCCCTGCAATATTTGCATG
TCGCTATGTGTTCTGGGAAATCACCATAAACGTGAAATGTCTTTGGATTT
GGGAATCTTATAAGTTCTGTATGAGACCACTCTTTCCC----TTTTTGGG
AAAAAAAAAAAAAAAAAAAAAACGAAACCGGGCCGGGCGCGGTGGTTCAC
GCCTATAATCCCAGCACTTTGGGAGGCCGAGGCGGGCGGATCACAAGGTC
AGGAGGTCGAGACCATCCAGGCTAACACGGTGAAACCCCCCCCCATCTCT
ACTAAAAAAAAAAAATACAAAAAATTAGCCATTAGCCGGGCGTGGTGGCG
GGCGCCTATAATCCCAGCTACTTGGGAGGCTGAAGCAGAATGGCGTGAAC
CCGGGAGGCGGACGTTGCAGTGAGCCGAGATCGCGCCGACTGCATTCCAG
CCTGGGCGACAGAGCGAGTCTCAAAAAAAAAACCGAGTGGAATGTGAAAA
GCTCCGTGAAACTGCAGAAACCCAAGCCGAATTCTGCAGATATCCATCAC
ACTGGCGGCCGCTCGAGTGAGGCGGAAAGAACCAGCTGGGGCTCTAGGGG
GTATCCCCACGCGCCCTGTAGCGGCGCATTAAGCGCGGCGGGTGTGGTGG
TTACGCGCAGCGTGACCGCTACACTTGCCAGCGCCCTAGCGCCCGCTCCT
TTCGCTTTCTTCCCTTCCTTTCTCGCCACGTTCGCCGGCTTTCCCCGTCA
AGCTCTAAATCGGGGGCTCCCTTTAGGGTTCCGATTTAGTGCTTTACGGC
ACCTCGACCCCAAAAAACTTGATTAGGGTGATGGTTCACGTAGTGGGCCA
TCGCCCTGATAGACGGTTTTTCGCCCTTTGACGTTGGAGTCCACGTTCTT
TAATAGTGGACTCTTGTTCCAAACTGGAACAACACTCAACCCTATCTCGG
TCTATTCTTTTGATTTATAAGGGATTTTGCCGATTTCGGCCTATTGGTTA
AAAAATGAGCTGATTTAACAAAAATTTAACGCGAATTAATTCTGTGGAAT
GTGTGTCAGTTAGGGTGTGGAAAGTCCCCAGGCTCCCCAGCAGGCAGAAG
TATGCAAAGCATGCATCTCAATTAGTCAGCAACCAGGTGTGGAAAGTCCC
CAGGCTCCCCAGCAGGCAGAAGTATGCAAAGCATGCATCTCAATTAGTCA
GCAACCATAGTCCCGCCCCTAACTCCGCCCATCCCGCCCCTAACTCCGCC
CAGTTCCGCCCATTCTCCGCCCCATGGCTGACTAATTTTTTTTATTTATG
CAGAGGCCGAGGCCGCCTCTGCCTCTGAGCTATTCCAGAAGTAGTGAGGA
GGCTTTTTTGGAGGCCTAGGCTTTTGCAAAAAGCTCCCGGGAGCTTGTAT
ATCCATTTTCGGATCTGATCAAGAGACAGGATGAGGATCGTTTCGCATGA
TTGAACAAGATGGATTGCACGCAGGTTCTCCGGCCGCTTGGGTGGAGAGG
CTATTCGGCTATGACTGGGCACAACAGACAATCGGCTGCTCTGATGCCGC
CGTGTTCCGGCTGTCAGCGCAGGGGCGCCCGGTTCTTTTTGTCAAGACCG
ACCTGTCCGGTGCCCTGAATGAACTGCAGGACGAGGCAGCGCGGCTATCG
TGGCTGGCCACGACGGGCGTTCCTTGCGCAGCTGTGCTCGACGTTGTCAC
TGAAGCGGGAAGGGACTGGCTGCTATTGGGCGAAGTGCCGGGGCAGGATC
TCCTGTCATCTCACCTTGCTCCTGCCGAGAAAGTATCCATCATGGCTGAT
GCAATGCGGCGGCTGCATACGCTTGATCCGGCTACCTGCCCATTCGACCA
CCAAGCGAAACATCGCATCGAGCGAGCACGTACTCGGATGGAAGCCGGTC
TTGTCGATCAGGATGATCTGGACGAAGAGCATCAGGGCTCGCGCCAGCCG
AACTGTTCGCCAGGCTCAAGGCGCGCATGCCCCGACGGCGAGGATCTCGT
CGTGACCCATGGCGATGCCTGCTTGCCGAATATCATGGTGGAAAATGGCC
GCTTTTCTGGATTCATCGACTGCGGCCGGCTGGGTGTGGCGGACCGCTAT
CAGGACATAGCGTTGGCTACCCGTGATATTGCTGAAGAGCTTGGCGGCGA
ATGGGCTGACCGCTTCCTCGTGCTTTACGGTATCGCCGCTCCCGATTCGC
AGCGCATCGCCTTCTATCGCCTTCTTGACGAGTTCTTCTGAGCGGGACTC
TGGGGTTCGAAATGACCGACCAAGCGACGCCCAACCTGCCATCACGAGAT
TTCGATTCCACCGCCGCCTTCTATGAAAGGTTGGGCTTCGGAATCGTTTT
CCGGGACGCCGGCTGGATGATCCTCCAGCGCGGGATCTCATGCTGGAGTT
CTTCGCCCACCCCAACTTGTTTATTGCAGCTTATAATGGTTACAAATAAA
GCAATAGCATCACAAATTTCACAAATAAAGCATTTTTTTCACTGCATTCT
AGTTGTGGTTTGTCCAAACTCATCAATGTATCTTATCATGTCTGTATACC
GTCGACCTCTAGCTAGAGCTTGGCGTAATCATGGTCATAGCTGTTTCCTG
TGTGAAATTGTTATCCGCTCACAATTCCACACAACATACGAGCCGGAAGC
ATAAAGTGTAAAGCCTGGGGTGCCTAATGAGTGAGCTAACTCACATTAAT
TGCGTTGCGCTCACTGCCCGCTTTCCAGTCGGGAAACCTGTCGTGCCAGC
TGCATTAATGAATCGGCCAACGCGCGGGGAGAGGCGGTTTGCGTATTGGG
CGCTCTTCCGCTTCCTCGCTCACTGACTCGCTGCGCTCGGTCGTTCGGCT
GCGGCGAGCGGTATCAGCTCACTCAAAGGCGGTAATACGGTTATCCACAG
AATCAGGGGATAACGCAGGAAAGAACATGTGAGCAAAAGGCCAGCAAAAG
GCCAGGAACCGTAAAAAGGCCGCGTTGCTGGCGTTTTTCCATAGGCTCCG
CCCCCCGACGAGCATCACAAAAATCGACGCTCAAGTCAGAGGTGGCGAAA
CCCGACAGGACTATAAAGATACCAGGCGTTTCCCCCTGGAAGCTCCCTCG
TGCGCTCTCCTGTTCCGACCCTGCCGCTTACCGGATACCTGTCCGCCTTT
CTCCCTTCGGGAAGCGTGGCGCTTTCTCATAGCTCACGCTGTAGGTATCT
CAGTTCGGTGTAGGTCGTTCGCTCCAAGCTGGGCTGTGTGCACGAACCCC
CCGTTCAGCCCGACCGCTGCGCCTTATCCGGTAACTATCGTCTTGAGTCC
AACCCGGTAAGACACGACTTATCGCCACTGGCAGCAGCCACTGGTAACAG
GATTAGCAGAGCGAGGTATGTAGGCGGTGCTACAGAGTTCTTGAAGTGGT
GGCCTAACTACGGCTACACTAGAAGAACAGTATTTGGTATCTGCGCTCTG
CTGAAGCCAGTTACCTTCGGAAAAAGAGTTGGTAGCTCTTGATCCGGCAA
ACAAACCACCGCTGGTAGCGGTTTTTTTGTTTGCAAGCAGCAGATTACGC
GCAGAAAAAAAGGATCTCAAGAAGATCCTTTGATCTTTTCTACGGGGTCT
GACGCTCAGTGGAACGAAAACTCACGTTAAGGGATTTTGGTCATGAGATT
ATCAAAAAGGATCTTCACCTAGATCCTTTTAAATTAAAAATGAAGTTTTA
AATCAATCTAAAGTATATATGAGTAAACTTGGTCTGACAGTTACCAATGC
TTAATCAGTGAGGCACCTATCTCAGCGATCTGTCTATTTCGTTCATCCAT
AGTTGCCTGACTCCCCGTCGTGTAGATAACTACGATACGGGAGGGCTTAC
CATCTGGCCCCAGTGCTGCAATGATACCGCGAGACCCACGCTCACCGGCT
CCAGATTTATCAGCAATAAACCAGCCAGCCGGAAGGGCCGAGCGCAGAAG
TGGTCCTGCAACTTTATCCGCCTCCATCCAGTCTATTAATTGTTGCCGGG
AAGCTAGAGTAAGTAGTTCGCCAGTTAATAGTTTGCGCAACGTTGTTGCC
ATTGCTACAGGCATCGTGGTGTCACGCTCGTCGTTTGGTATGGCTTCATT
CAGCTCCGGTTCCCAACGATCAAGGCGAGTTACATGATCCCCCATGTTGT
GCAAAAAAGCGGTTAGCTCCTTCGGTCCTCCGATCGTTGTCAGAAGTAAG
TTGGCCGCAGTGTTATCACTCATGGTTATGGCAGCACTGCATAATTCTCT
TACTGTCATGCCATCCGTAAGATGCTTTTCTGTGACTGGTGAGTACTCAA
CCAAGTCATTCTGAGAATAGTGTATGCGGCGACCGAGTTGCTCTTGCCCG
GCGTCAATACGGGATAATACCGCGCCACATAGCAGAACTTTAAAAGTGCT
CATCATTGGAAAAACGTTCTTCGGGGCGAAAACTCTCAAGGATCTTACCG
CTGTTGAGATCCAGTTCGATGTAACCCACTCGTGCACCCAACTGATCTTC
AGCATCTTTTACTTTCACCAGCGTTTCTGGGTGAGCAAAAACAGGAAGGC
AAAATGCCGCAAAAAAGGGAATAAGGGCGACACGGAAATGTTGAATACTC
ATACTCTTCCTTTTTCAATATTATTGAAGCATTTATCAGGGTTATTGTCT
CATGAGCGGATACATATTTGAATGTATTTAGAAAAATAAACAAATAGGGG
TTCCGCGCACATTTCCCCGAAAAGTGCCACCTGACGTC

The nucleotide sequences of the hairpin loop structure and target sequence of the siRNA is shown in SEQ ID NO:22 and SEQ ID NO:21, respectively (endonuclease recognition sites are eliminated from each hairpin loop structure sequence). A control plasmid, psiH1BX-EGFP was prepared by cloning double-stranded oligonucleotides of

(SEQ ID No; 17)

5′-CACCGAAGCAGCACGACTTCTTCTTCAAGAGAGAAGAAGTCGTGCTG
CTTC-3′
and

(SEQ ID No; 18)

5′-AAAAGAAGCAGCACGACTTCTTCTCTCTTGAAGAAGAAGTCGTGCTG
CTTC-3′

into the BbsI site in the psiHIBX3.0 vector. Plasmids expressing C10orf3-siRNA were prepared by cloning of double-stranded oligonucleotides into psiH1BX3.0 vector. The oligonucleotides used for C10orf3-siRNA were

(SEQ ID No; 19)

5′-TCCCGGAGAGACTGAAAACAGAGTTCAAGAGACTCTGTTTTCAGTCT
CTCC-3′
and

(SEQ ID No; 20)

5′-AAAAGGAGAGACTGAAAACAGAGTCTCTTGAACTCTGTTTTCAGTCT
CTCC-3′
(psiH1BX-C10orf3-g).

### Cell Viability Assay

SW480 and HCT116 cells were transfected with psiH1BX-C10orf3E, psiH1BX-C10orf3G or control plamids (psiH1BX-EGFP) and maintained in the culture media in the presence of 454 μg/ml geneticin. Ten days after transfection, the number of viable cells were measured by MTT assay. Subsequently, the absorbance of cell lysate was measured with a spectrophoto plate reader at a test wavelength of 450 nm (reference, 600 nm). The cell viability was represented by the absorbance compared to that of control cells.

### EXAMPLE 2

### Identification of C10orf3, a Up-Regulated Gene, in Colorectal Cancers

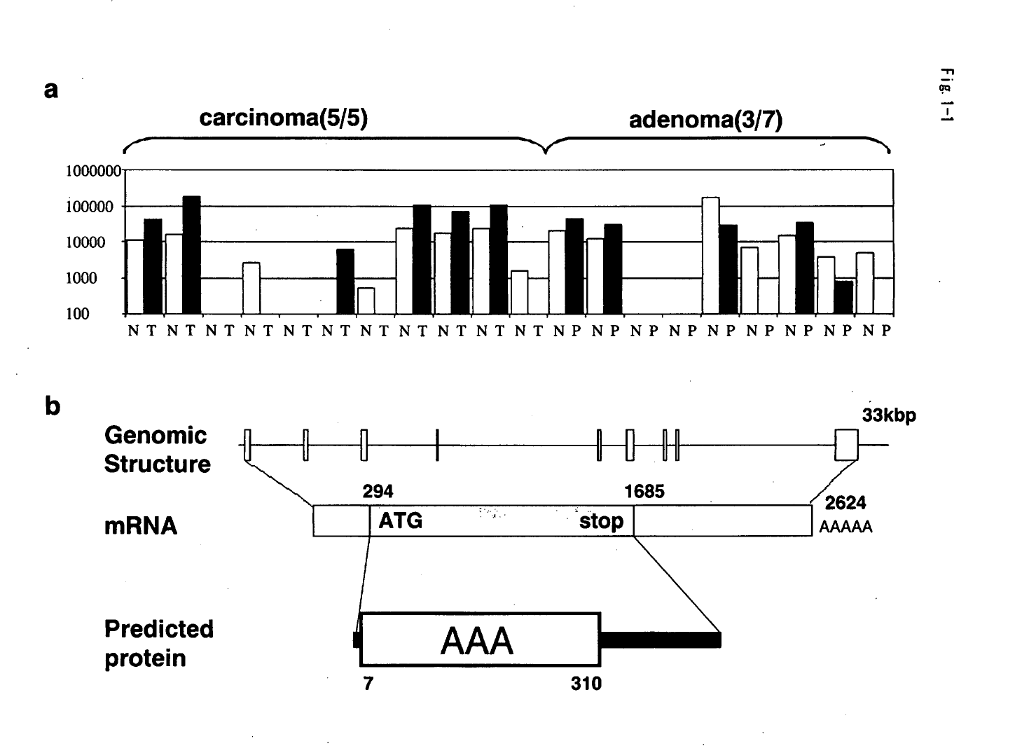

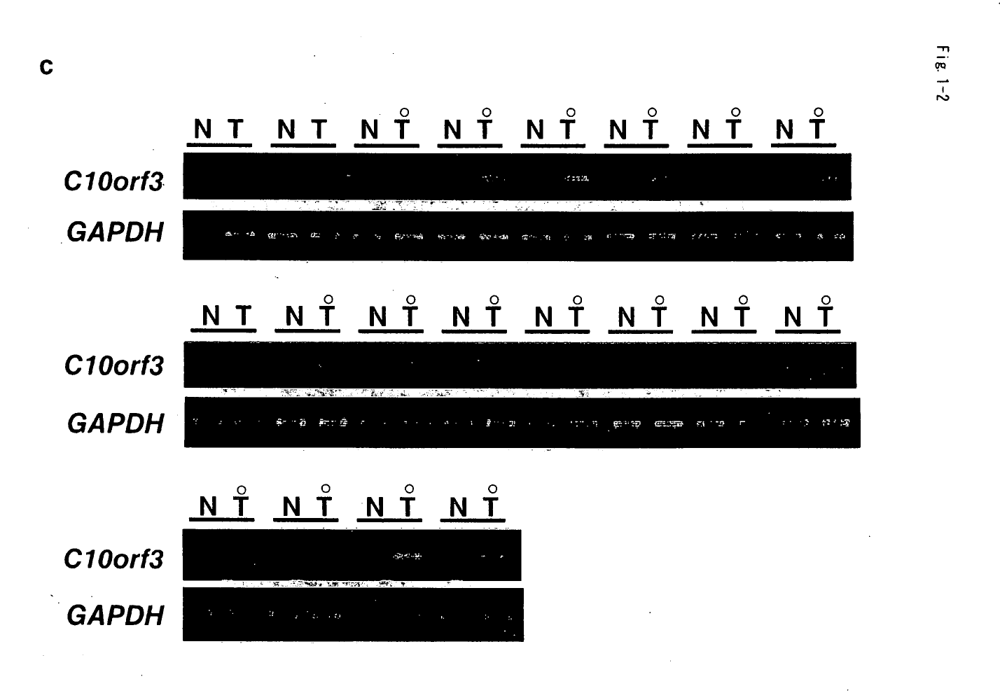

We also compared expression profiles of 11 colorectal cancers and 9 colonic adenomas with the corresponding non-cancerous colon tissues using the cDNA microarray containing 23040 genes. Among commonly up-regulated genes in colorectal cancers, a gene with an in-house accession number of B5904, corresponding to an EST Hs. 14559 of a UniGene cluster (http://www.ncbi.nlm.nih.gov/UniGene/), was over-expressed in all five CRCs and 3 out of 4 adenomas with valid signal intensities compared with the corresponding noncancerous colon tissues (FIG. 1a). A homology search in public databases with the B5904 nucleotide sequence revealed that B5904 is identical to C10orf3, that contained 2624 nucleotides (GenBank accession number of AB091343) encoding a putative 464-amino acid protein with a predicted molecular mass of 54 kD. Comparison of the assembled cDNA sequence with the genomic sequence allowed us to determine that the gene consisted of 9 exons and covered a genomic region of approximately 33 kb at chromosomal band 10q23 (FIG. 1b). A search for protein motifs with Simple Modular Architecture Research Tool (SMART, http://smart.embl-heidelberg.de) revealed that the predicted protein contained an AAA (ATPase-associated with variety cellular activities) domain (FIG. 1b). We further analyzed expression of C10orf3 by semi-quantitative RT-PCR using additional 20 CRC cases, and observed its elevated expression in 17 out of the 20 cancers compared with their non-cancerous colon tissues (FIG. 1c).

### EXAMPLE 3

### Expression of C10orf3 in Normal Adult Tissues

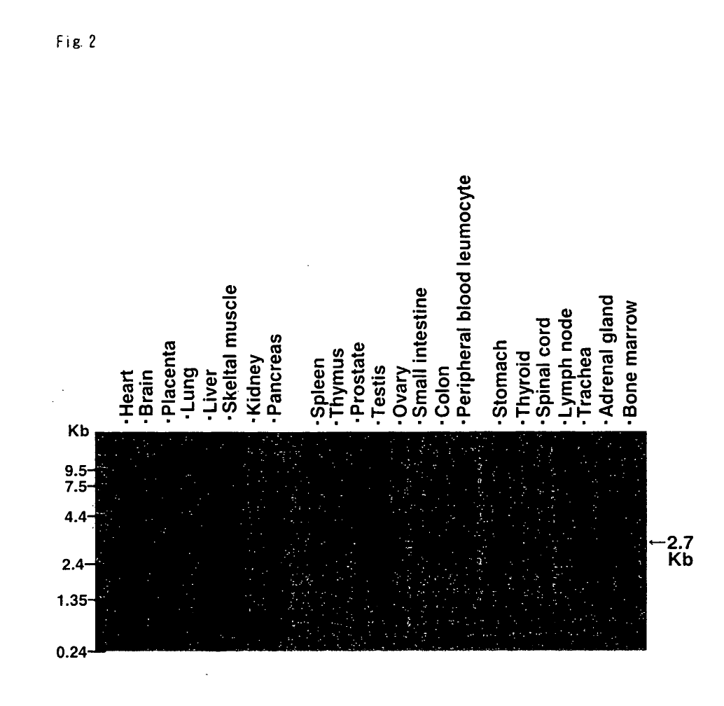

Multiple-tissue northern blot analysis using a PCR product of C10orf3 as a probe detected a 2.7-kb transcript abundantly expressed in testis and slightly expressed in small intestine, colon, stomach, placenta, and ovary (FIG. 2).

### EXAMPLE 4

### Expression of C10orf3 Protein in Colon Cancer

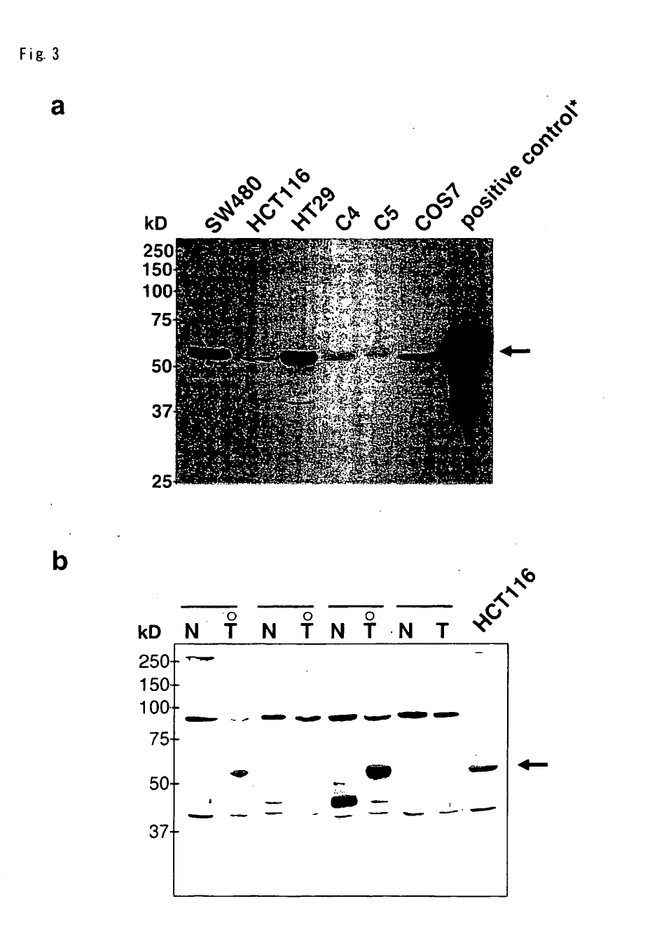

To examine the expression and explore the function of C10orf3, we prepared polyclonal antibody against C10orf3. Western blot analysis using whole extracts of COS7 and colon cancer cells, including SW480, HCT116, HT29, SNUC4 and SNUC5 showed a 54 kDa-band that corresponded to C10orf3 (FIG. 3a). The size of endogenous C10orf3 protein was quite similar to that of flag-tagged C10orf3 detected with anti-C10orf3 antibody. An additional western blot analysis using extracts from four cancer tissues and their corresponding non-cancerous mucosa revealed its elevated expression in cancer in three cases among the four (FIG. 3b).

### EXAMPLE 5

### Subcellular Localization of C10orf3

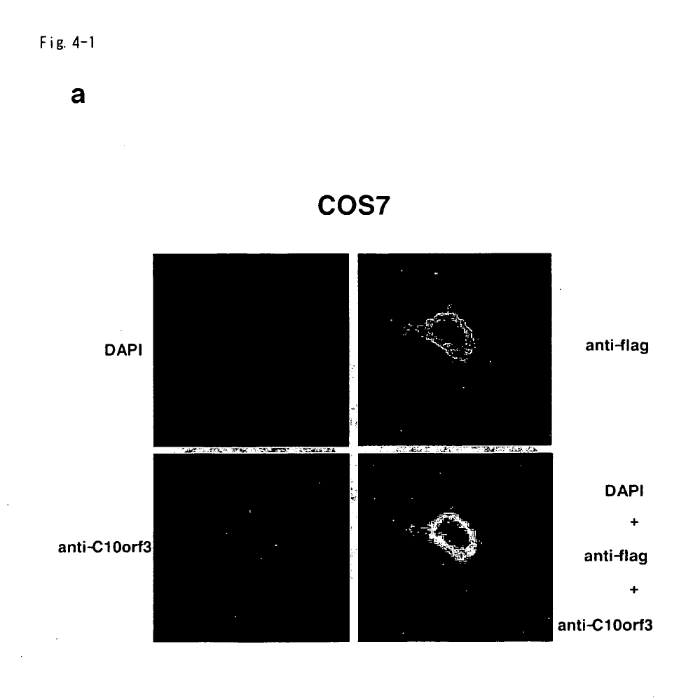

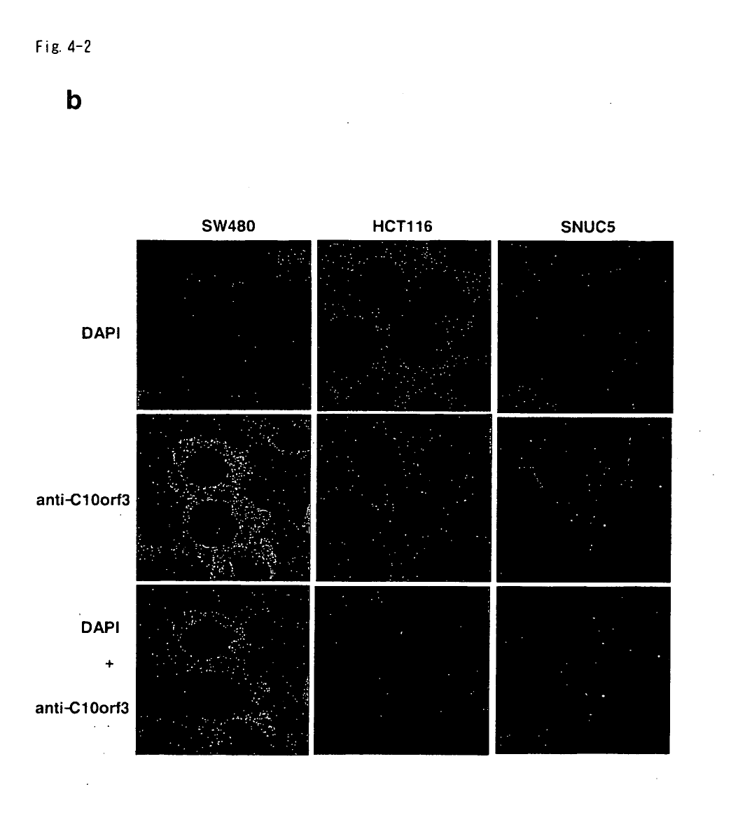

To investigate the sublocalization of C10orf3 protein, we performed fluorescent immunohistochemical staining using anti-C10orf3 antibody. COS7 cells transfected with pFLAG-CMV-5a-C10orf3 were co-stained with anti-Flag antibody and anti-C10orf3 antibody. As a result, both antibodies showed the same subcellular localization of exogeneous Flag-tagged C10orf3 protein in the cytoplasm (FIG. 4a). We also performed immunocytochemical staining using SW480, HCT116 and SNUC5 colon cancer cells to examine endogenous C10orf3 protein. Consistently, the analysis demonstrated the same subcellular localization of C10orf3 in the cytoplasm in these three cell lines (FIG. 4b).

### EXAMPLE 6

### Growth Suppression of Colon Cancer Cells by C10orf3 siRNA

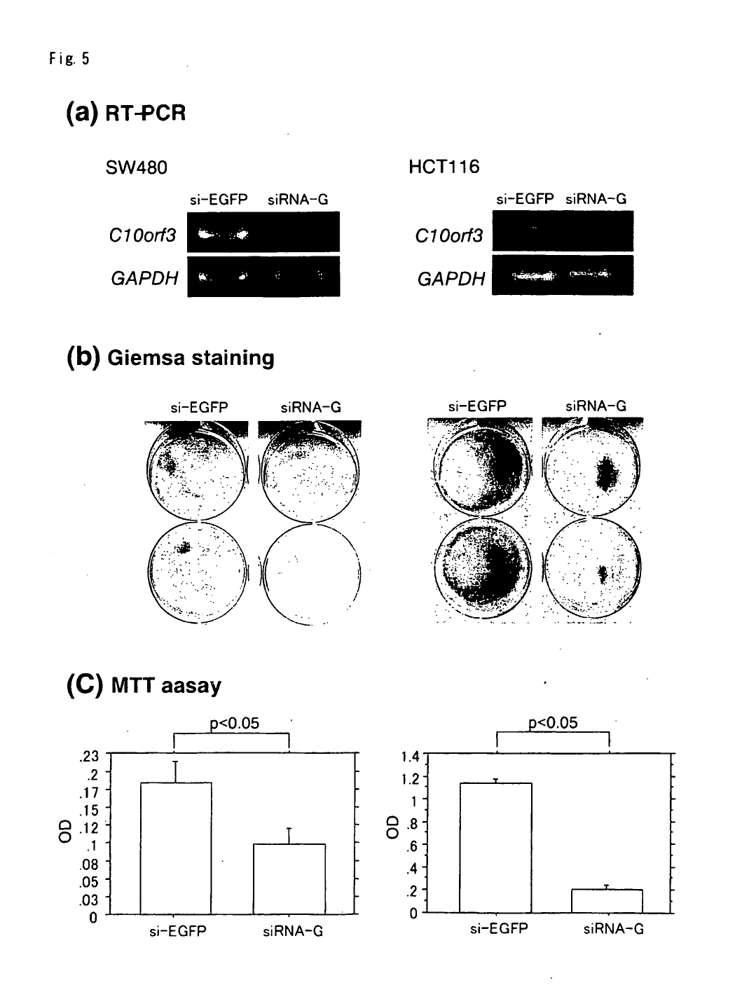

To examine the function of C10orf3 in cell growth, we transfected SW480 and HCT116 cells with plasmids expressing siRNA to C10orf3. As a result, semi-quantitative RT-PCR showed that psiH1BX-C10orf3-G significantly suppressed the expression of C10orf3 compared with the control plasmid (psiH1BX-EGFP) (FIG. 5a). At day 10 after the transfection, the number of viable cells transfected with psiH1BX-C10orf3-G was markedly decreased compared with that psiH1BX-EGFP (FIG. 5b, c). The growth inhibitory effect of the plasmids was well correlated to their gene silencing activity. This result suggests that C10orf3 may play an important role for cell growth and/or survival in colorectal cancer cells.

### INDUSTRIAL APPLICABILITY

The previous gene-expression analysis of genome-wide cDNA microarray has identified specific up-regulated gene C10orf3. The present invention revealed C10orf3 serves as target for cancer prevention and therapy. Based on the expression of C10orf3, the present invention provides a molecular diagnostic marker for identifying or detecting CRC.

The methods described herein are also useful in the identification of additional molecular targets for prevention, diagnosis and treatment of CRC. The data reported herein add to a comprehensive understanding of CRC, facilitate development of novel diagnostic strategies, and provide clues for identification of molecular targets for therapeutic drugs and preventative agents. Such information contributes to a more profound understanding of colorectal tumorigenesis, and provides indicators for developing novel strategies for diagnosis, treatment, and ultimately prevention of CRC.

All patents, patent applications, and publications cited herein are incorporated by reference in their entirety. Furthermore, while the invention has been described in detail and with reference to specific embodiments thereof, it will be apparent to one skilled in the art that various changes and modifications can be made therein without departing from the spirit and scope of the invention.

### REFERENCES

1. Murthy, R. S., Bertolote, J. M., Epping-Jordan, J., Funk, M., Prentice, T., Saraceno, B., and Saxena, S. The World Health Report 2001. A. Haden and B. Campanini (eds.), pp. 144-149. Basel, Switzerland: World Health Organization, 2001.
2. Nishisho, I., Nakamura, Y., Miyoshi, Y., Miki, Y., Ando, H., Horii, A., Koyama, K., Utsunomiya, J., Baba, S., and Hedge, P. Mutation of chromosome 5q21 genes in FAP and colorectal cancer patients. Science (Wash. DC), 253:665-669, 1991.
3. Baker, S. J., Fearon, E. R., Nigro, J. M., Hamilton, S. R., Preisinger, A. C., Jessup, J. M., vanTuinen, P., Ledbetter, D. H., Barker, D. F., Nakamura, Y., White, R., and Vogelstein, B. Chromosome 17 deletions and p53 gene mutations in colorectal carcinomas. Science (Wash. DC), 244:217-221, 1989.
4. Morin, P. J., Sparks, A. B., Korinek, V., Barker, N., Clevers, H., Vogelstein, B., and Kinzler, K. W. Activation of beta-catenin-Tcf signaling in colon cancer by mutations in beta-catenin or APC. Science (Wash. DC), 275:1787-1790, 1997.
5. Forrester, K., Almoguera, C., Han, K., Grizzle, W. E., and Perucho, M. Detection of high incidence of K-ras oncogenes during human colon tumorigenesis. Nature (Lond.), 327: 298-303, 1987.
6. Jones, P. A., and Laird, P. W. Cancer epigenetics comes of age. Nat. Genet., 21:163-167, 1999.
7. Cui, H., Horon, I. L., Ohlsson, R., Hamilton, S. R., and Feinberg, A. P. Loss of imprinting in normal tissue of colorectal cancer patients with microsatellite instability. Nat. Med., 4: 1276-1280, 1998.
8. Molina, M. A., Codony-Servat, J., Albanell, J., Rojo, F., Arribas, J., and Baselga, J. Trastuzumab (herceptin), a humanized anti-Her2 receptor monoclonal antibody, inhibits basal and activated Her2 ectodomain cleavage in breast cancer cells. Cancer Res., 61: 4744-4749, 2001.
9. O'Dwyer, M. E., and Druker, B. J. Status of bcr-abl tyrosine kinase inhibitors in chronic myelogenous leukemia. Curr. Opin. Oncol., 12:594-597, 2000.
10. Afshari C A, Nuwaysir E F and Barrett J C. (1999). Cancer Res., 59, 4759-4760.
11. Kallioniemi O P. (2001). Ann. Med, 33, 142-147.
12. Ono K, Tanaka T, Tsunoda T, Kitahara 0, Kihara C, Okamoto A, Ochiai K, Takagi T and Nakamura Y. (2000). Cancer Res., 60, 5007-5011.
13. Kitahara O, Furukawa Y, Tanaka T, Kihara C, Ono K, Yanagawa R, Nita, M, Takagi T, Nakamura Y and Tsunoda T. (2001). Cancer Res., 61, 3544-3549.
14. Okabe H, Satoh S, Kato T, Kitahara O, Yanagawa R, Yamaoka Y, Tsunoda T, Furukawa Y and Nakamura. (2001). Cancer Res., 61, 2129-2137.

1. A method of diagnosing CRC or a predisposition to developing CRC in a subject, comprising determining a level of expression of C10orf3 in a patient derived biological sample, wherein an increase of said level compared to a normal control level of said gene indicates that said subject suffers from or is at risk of developing CRC.

2. The method of claim 1, wherein said increase is at least 10% greater than said normal control level.

3. The method of claim 1, wherein the expression level is determined by any one method selected from group consisting of:
(a) detecting the mRNA of C10orf3,
(b) detecting the protein encoded by C10orf3, and
(c) detecting the biological activity of the protein encoded by C10orf3,

4. The method of claim 1, wherein said level of expression is determined by detecting hybridization of C10orf3 probe to a gene transcript of said patient-derived biological sample.

5. The method of claim 4, wherein said hybridization step is carried out on a DNA array.

6. The method of claim 1, wherein said biological sample comprises an epithelial cell.

7. The method of claim 1, wherein said biological sample comprises CRC cell.

8. The method of claim 4, wherein said biological sample comprises an epithelial cell from a CRC.

9. A method of screening for a compound for treating or preventing CRC, said method comprising the steps of:
a) contacting a test compound with a polypeptide encoded by a nucleic acid of C10orf3;
b) detecting the binding activity between the polypeptide and the test compound; and
c) selecting a compound that binds to the polypeptide.

10. A method of screening for a compound for treating or preventing CRC, said method comprising the steps of:
a) contacting a candidate compound with a cell expressing C10orf3, and
b) selecting a compound that reduces the expression level of C10orf3.

11. The method of claim 10, wherein said cell comprises a colorectal cancer cell.

12. A method of screening for a compound for treating or preventing CRC, said method comprising the steps of:
a) contacting a test compound with a polypeptide encoded by a nucleic acid of C10orf3;
b) detecting the biological activity of the polypeptide of step (a); and
c) selecting a compound that suppresses the biological activity of the polypeptide encoded by a nucleic acid of C10orf3 in comparison with the biological activity detected in the absence of the test compound.

13. The method of claim 12, wherein the biological activity of the polypeptide is cell proliferative activity.

14. The method of claim 12, wherein the biological activity of the polypeptide is ATP-ase activity.

15. A method of screening for compound for treating or preventing CRC, said method comprising the steps of:
a) contacting a candidate compound with a cell into which a vector comprising the transcriptional regulatory region of C10orf3 and a reporter gene that is expressed under the control of the transcriptional regulatory region has been introduced
b) measuring the activity of said reporter gene; and
c) selecting a compound that reduces the expression level of said reporter gene, as compared to a control.

16. A kit comprising a detection reagent which binds to nucleic acid sequence or polypeptide of C10orf3.

17. A method of treating or preventing CRC in a subject comprising administering to said subject an antisense composition, said composition comprising a nucleotide sequence complementary to a coding sequence of C10orf3.

18. A method of treating or preventing CRC in a subject comprising administering to said subject a siRNA composition, wherein said composition reduces the expression of a nucleic acid sequence of C10orf3.

19. The method of claim 18, wherein the siRNA comprises a sense strand comprising the nucleotide sequence of SEQ ID NO: 21 as the target sequence.

20. The method of claim 19, said siRNA has the general formula 5′-[A]-[B]-[A′]-3′, wherein [A] is a ribonucleotide sequence corresponding to the nucleotide sequence of SEQ ID NO: 21,
[B] is a ribonucleotide sequence consisting of 3 to 23 nucleotides, and [A′] is a ribonucleotide sequence consisting of the complementary sequence of [A].

21. The method of claim 18, wherein said composition comprises a transfection-enhancing agent.

22. A method for treating or preventing CRC in a subject comprising the step of administering to said subject a pharmaceutically effective amount of an antibody or fragment thereof that binds to a protein encoded by nucleic acid of C10orf3.

23. A method of treating or preventing CRC in a subject comprising administering to said subject a vaccine comprising a polypeptide encoded by a nucleic acid of C10orf3 or an immunologically active fragment of said polypeptide, or a polynucleotide encoding the polypeptide.

24. A method for treating or preventing CRC in a subject, said method comprising the step of administering a compound that is obtained by the method according to any one of claims 9-15.

25. A composition for treating or preventing CRC, said composition comprising a pharmaceutically effective amount of an antisense polynucleotide or small interfering RNA against a polynucleotide of C10orf3 as an active ingredient, and a pharmaceutically acceptable carrier.

26. The composition of claim 25, wherein the siRNA comprises a sense strand comprising the nucleotide sequence of SEQ ID NO: 21 as the target sequence.

27. The composition of claim 26, said siRNA has the general formula 5′-[A]-[B]-[A′]-3′, wherein [A] is a ribonucleotide sequence corresponding to the nucleotide sequence of SEQ ID NO: 21,
[B] is a ribonucleotide sequence consisting of 3 to 23 nucleotides, and
[A′] is a ribonucleotide sequence consisting of the complementary sequence of [A].

28. A composition for treating or preventing CRC, said composition comprising a pharmaceutically effective amount of an antibody or fragment thereof that binds to a protein encoded by nucleic acid of C10orf3 as an active ingredient, and a pharmaceutically acceptable carrier.

29. A composition for treating or preventing CRC, said composition comprising a pharmaceutically effective amount of the compound selected by the method of any one of claims 9-15 as an active ingredient, and a pharmaceutically acceptable carrier.

30. A double-stranded molecule comprising a sense strand and an antisense strand, wherein the sense strand comprises a ribonucleotide sequence corresponding to SEQ ID NO: 21, and wherein the antisense strand comprises a ribonucleotide sequence which is complementary to said sense strand, wherein said sense strand and said antisense strand hybridize to each other to form said double-stranded molecule, and wherein said double-stranded molecule, when introduced into a cell expressing the C10orf3 gene, inhibits expression of said gene.

31. The double-stranded molecule of claim 30, wherein said sense strand comprises from about 19 to about 25 contiguous nucleotides from SEQ ID No:1.

32. The double-stranded molecule of claim 30, wherein said sense strand consists of the ribonucleotide sequence corresponding to SEQ ID NO: 21.

33. The double-stranded molecule of claim 30, wherein a single ribonucleotide transcript comprises the sense strand and the antisense strand, said double-stranded molecule further comprising a single-stranded ribonucleotide sequence linking said sense strand and said antisense strand.

34. A vector encoding the double-stranded molecule of claim 30.

35. The vector of claim 34, wherein the vector encodes a transcript having a secondary structure, wherein the transcript comprises the sense strand and the anti sense strand.

36. The vector of claim 34, wherein the transcript further comprises a single-stranded ribonucleotide sequence linking said sense strand and said antisense strand.


SEQUENCE LISTING (26 sequences)

SEQ ID NO: 1 - DNA, 2624 bp, Homo sapiens
  C10orf3
ggcacgaggggccgacgcgagcgccgcgcttcgcttcagctgctagctggcccaagggaggcgaccgcggagggtggcgaggggcggccaggacccgcagccccggggccgggccggtccggaccgccagggagggcaggtcagtgggcagatcgcgtccgcgggattcaatctctgcccgctctgataacagtccttttccctggcgctcacttcgtgcctggcacccggctgggcgcctcaagaccgttgtctcttcgatcgcttctttggacttggcgaccatttcagagatgtcttccagaagtaccaaagatttaattaaaagtaagtggggatcgaagcctagtaactccaaatccgaaactacattagaaaaattaaagggagaaattgcacacttaaagacatcagtggatgaaatcacaagtgggaaaggaaagctgactgataaagagagacacagacttttggagaaaattcgagtccttgaggctgagaaggagaagaatgcttatcaactcacagagaaggacaaagaaatacagcgactgagagaccaactgaaggccagatatagtactaccgcattgcttgaacagctggaagagacaacgagagaaggagaaaggagggagcaggtgttgaaagccttatctgaagagaaagacgtattgaaacaacagttgtctgctgcaacctcacgaattgctgaacttgaaagcaaaaccaatacactccgtttatcacagactgtggctccaaactgcttcaactcatcaataaataatattcatgaaatggaaatacagctgaaagatgctctggagaaaaatcagcagtggctcgtgtatgatcagcagcgggaagtctatgtaaaaggacttttagcaaagatctttgagttggaaaagaaaacggaaacagctgctcattcactcccacagcagacaaaaaagcctgaatcagaaggttatcttcaagaagagaagcagaaatgttacaacgatctcttggcaagtgcaaaaaaagatcttgaggttgaacgacaaaccataactcagctgagttttgaactgagtgaatttcgaagaaaatatgaagaaacccaaaaagaagttcacaatttaaatcagctgttgtattcacaaagaagggcagatgtgcaacatctggaagatgataggcataaaacagagaagatacaaaaactcagggaagagaatgatattgctaggggaaaacttgaagaagagaagaagagatccgaagagctcttatctcaggtccagtttctttacacatctctgctaaagcagcaagaagaacaaacaagggtagctctgttggaacaacagatgcaggcatgtactttagactttgaaaatgaaaaactcgaccgtcaacatgtgcagcatcaattgcatgtaattcttaaggagctccgaaaagcaagaaatcaaataacacagttggaatccttgaaacagcttcatgagtttgccatcacagagccattagtcactttccaaggagagactgaaaacagagaaaaagttgccgcctcaccaaaaagtcccactgctgcactcaatgaaagcctggtggaatgtcccaagtgcaatatacagtatccagccactgagcatcgcgatctgcttgtccatgtggaatactgttcaaagtagcaaaataagtatttgttttgatattaaaagattcaatactgtattttctgttagcttgtgggcattttgaattatatatttcacattttgcataaaactgcctatctacctttgacactccagcatgctagtgaatcatgtatcttttaggctgctgtgcatttctcttggcagtgatacctccctgacatggttcatcatcaggctgcaatgacagaatgtggtgagcagcgtctactgagactactaacattttgcactgtcaaaatacttggtgaggaaaagatagctcaggttattgctaatgggttaatgcaccagcaagcaaaatattttatgttttgggggtttgaaaaatcaaagataattaaccaaggatcttaactgtgttcgcattttttatccaagcacttagaaaacctacaatcctaattttgatgtccattgttaagaggtggtgatagatactattttttttttcatattgtatagcggttattagaaaagttggggattttcttgatctttattgctgcttaccattgaaacttaacccagctgtgttccccaactctgttctgcgcacgaaacagtatctgtttgaggcataatcttaagtggccacacacaatgttttctcttatgttatctggcagtaactgtaacttgaattacattagcacattctgcttagctaaaattgttaaaataaactttaataaacccatgtagccctctcatttgattgacagtattttagttatttttggcattcttaaagctgggcaatgtaatgatcagatctttgtttgtctgaacaggtatttttatacatgctttttgtaaaccaaaaacttttaaatttcttcaggttttctaacatgcttaccactgggctactgtaaatgagaaaagaataaaattatttaatgtttt

SEQ ID NO: 2 - protein, 464 aa, Homo sapiens
  C10orf3
MSSRSTKDLIKSKWGSKPSNSKSETTLEKLKGEIAHLKTSVDEITSGKGKLTDKERHRLLEKIRVLEAEKEKNAYQLTEKDKEIQRLRDQLKARYSTTALLEQLEETTREGERREQVLKALSEEKDVLKQQLSAATSRIAELESKTNTLRLSQTVAPNCFNSSINNIHEMEIQLKDALEKNQQWLVYDQQREVYVKGLLAKIFELEKKTETAAHSLPQQTKKPESEGYLQEEKQKCYNDLLASAKKDLEVERQTITQLSFELSEFRRKYEETQKEVHNLNQLLYSQRRADVQHLEDDRHKTEKIQKLREENDIARGKLEEEKKRSEELLSQVQFLYTSLLKQQEEQTRVALLEQQMQACTLDFENEKLDRQHVQHQLHVILKELRKARNQITQLESLKQLHEFAITEPLVTFQGETENREKVAASPKSPTAALNESLVECPKCNIQYPATEHRDLLVHVEYCSK

SEQ ID NO: 3 - DNA, 22 bp, Artificial Sequence
  GAPDH RT-PCR amplification forward primer
acaacagcctcaagatcatcag

SEQ ID NO: 4 - DNA, 20 bp, Artificial Sequence
  GAPDH RT-PCR amplification reverse primer
ggtccaccactgacacgttg

SEQ ID NO: 5 - DNA, 23 bp, Artificial Sequence
  C10orf3 RT-PCR amplification forward primer
agagatccgaagagctcttatct

SEQ ID NO: 6 - DNA, 21 bp, Artificial Sequence
  C10orf3 RT-PCR amplification reverse primer
gatgctcagtggctggatact

SEQ ID NO: 7 - DNA, 24 bp, Artificial Sequence
  C10orf3 gene-specific RT-PCR amplification forward primer
cgaaagcttcagagatgtcttcca

SEQ ID NO: 8 - DNA, 27 bp, Artificial Sequence
  C10orf3 gene-specific RT-PCR amplification reverse primer
aatggatccctttgaacagtattccac

SEQ ID NO: 9 - DNA, 26 bp, Artificial Sequence
  C10orf3 RT-PCR product forward primer for cloning into pET28a vector
atagaattcatgtcttccagaagtac

SEQ ID NO: 10 - DNA, 22 bp, Artificial Sequence
  C10orf3 RT-PCR product reverse primer for cloning into pET28a vector
tatctcgagctttgaacagtat

SEQ ID NO: 11 - DNA, 22 bp, Artificial Sequence
  H1RNA gene containing promoter region genomic fragment PCR amplification primer for siRNA plasmid vector
tggtagccaagtgcaggttata

SEQ ID NO: 12 - DNA, 22 bp, Artificial Sequence
  H1RNA gene containing promoter region genomic fragment PCR amplification primer for siRNA plasmid vector
ccaaagggtttctgcagtttca

SEQ ID NO: 13 - DNA, 30 bp, Artificial Sequence
  BamHI and XhoI fragment containing H1RNA PCR amplification primer
tgcggatccagagcagattgtactgagagt

SEQ ID NO: 14 - DNA, 29 bp, Artificial Sequence
  BamHI and XhoI fragment containing H1RNA PCR amplification primer
ctctatctcgagtgaggcggaaagaacca

SEQ ID NO: 15 - DNA, 47 bp, Artificial Sequence
  PCR amplification primer for ligated DNA template
tttaagcttgaagaccatttttggaaaaaaaaaaaaaaaaaaaaaac

SEQ ID NO: 16 - DNA, 34 bp, Artificial Sequence
  PCR amplification primer for ligated DNA template
tttaagcttgaagacatgggaaagagtggtctca

SEQ ID NO: 17 - DNA, 51 bp, Artificial Sequence
  double-stranded oligonucleotide for cloning control plasmid psiH1BX-EGFP
caccgaagcagcacgacttcttcttcaagagagaagaagtcgtgctgcttc

SEQ ID NO: 18 - DNA, 51 bp, Artificial Sequence
  double-stranded oligonucleotide for cloning control plasmid psiH1BX-EGFP
aaaagaagcagcacgacttcttctctcttgaagaagaagtcgtgctgcttc

SEQ ID NO: 19 - DNA, 51 bp, Artificial Sequence
  double-stranded oligonucleotide for cloning into psiH1BX3.0 vector plasmid expressing C10orf3-siRNA (psiH1BX-C10orf3-G)
tcccggagagactgaaaacagagttcaagagactctgttttcagtctctcc

SEQ ID NO: 20 - DNA, 51 bp, Artificial Sequence
  double-stranded oligonucleotide for cloning into psiH1BX3.0 vector plasmid expressing C10orf3-siRNA (psiH1BX-C10orf3-G)
aaaaggagagactgaaaacagagtctcttgaactctgttttcagtctctcc

SEQ ID NO: 21 - DNA, 19 bp, Artificial Sequence
  C10orf3 siRNA oligonucleotide target sequence
ggagagactgaaaacagag

SEQ ID NO: 22 - DNA, 47 bp, Artificial Sequence
  C10orf3 siRNA oligonucleotide hairpin loop structure
ggagagactgaaaacagagttcaagagactctgttttcagtctctcc

SEQ ID NO: 23 - DNA, 488 bp, Artificial
  psiH1 BX3.0 vector plasmid sequence upstream of siRNA DNA fragment insertion site
gacggatcgggagatctcccgatcccctatggtgcactctcagtacaatctgctctggatccactagtaacggccgccagtgtgctggaattcggcttggtagccaagtgcaggttatagggagctgaagggaagggggtcacagtaggtggcatcgttcctttctgactgcccgccccccgcatgccgtcccgcgatattgagctccgaacctctcgccctgccgccgccggtgctccgtcgccgccgcgccgccatggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttccc

SEQ ID NO: 24 - DNA, 4597 bp, Artificial
  psiH1 BX3.0 vector plasmid sequence downstream of siRNA DNA fragment insertion site
tttttgggaaaaaaaaaaaaaaaaaaaaaacgaaaccgggccgggcgcggtggttcacgcctataatcccagcactttgggaggccgaggcgggcggatcacaaggtcaggaggtcgagaccatccaggctaacacggtgaaacccccccccatctctactaaaaaaaaaaaatacaaaaaattagccattagccgggcgtggtggcgggcgcctataatcccagctacttgggaggctgaagcagaatggcgtgaacccgggaggcggacgttgcagtgagccgagatcgcgccgactgcattccagcctgggcgacagagcgagtctcaaaaaaaaaaccgagtggaatgtgaaaagctccgtgaaactgcagaaacccaagccgaattctgcagatatccatcacactggcggccgctcgagtgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 25 - RNA, 10 nt, Artificial Sequence
  2-10 u nucleotides added to 3′ end of antisense strand of target sequence to enhance siRNA inhibition activity
uuuuuuuuuu

SEQ ID NO: 26 - DNA, 18 bp, Artificial Sequence
  poly dT-12-18 primer
tttttttttttttttttt